(19) United States
(12) Patent Application Publication
(10) Pub. No.: US 2016/0022571 A1
(43) Pub. Date: Jan. 28, 2016

(54) IMPLANTABLE DRUG DELIVERY COMPOSITIONS COMPRISING NON-POLYMERIC SORPTION ENHANCERS AND METHODS OF TREATMENT THEREOF

(71) Applicant: ENDO PHARMACEUTICALS SOLUTIONS INC., Malvern, PA (US)

(72) Inventors: Alexander SCHWARZ, Brookline, MA (US); Denita WINSTEAD, New Hope, PA (US)

(73) Assignee: BRAEBURN PHARMACEUTICALS BVBA SPRL, BRUSSELS (BE)

(21) Appl. No.: 14/775,243

(22) PCT Filed: Mar. 13, 2014
(86) PCT No.: PCT/US14/25937

§ 371 Date: Sep. 11, 2015

Related U.S. Application Data
(60) Provisional application No. 61/784,038, filed on Mar. 14, 2013.
(60) Provisional application No. 61/815,366, filed on Apr. 24, 2013.

(51) Int. Cl. A61K 9/00; A61K 31/4535; A61K 47/18; A61K 47/34; A61K 31/428; A61K 31/53; A61K 47/22; A61K 47/02; A61K 47/12; A61K 31/4196; A61K 31/5685; A61K 31/136; A61K 31/433; A61K 31/137; A61K 31/167; A61K 38/09; A61K 31/519

(52) U.S. Cl. CPC A61K 9/0024; A61K 31/519; A61K 31/4535; A61K 47/183; A61K 47/34; A61K 31/428; A61K 31/53; A61K 47/22; A61K 47/02; A61K 47/12; A61K 31/4196; A61K 31/5685; A61K 31/136; A61K 31/433; A61K 31/137; A61K 31/167; A61K 38/09

(57) ABSTRACT

A drug delivery composition comprises a drug elution rate-controlling excipient comprising an elastomeric polymer defining a reservoir. The reservoir contains at least one discrete solid dosage form comprising at least one active pharmaceutical ingredient (API) and one or more non-polymeric sorption enhancers (e.g., one or more non-polymeric acids, bases, or salts). The drug delivery composition is in an implantable dosage form. A method of treating or preventing one or more diseases or conditions in a subject comprises implanting the drug delivery composition into a subject to systemically deliver a therapeutically effective amount of the API to the subject for a period of time of at least one month at a pseudo-zero order elution rate.

This application is related to and claims the benefit of U.S. Provisional Application No. 61/784,038, entitled “IMPLANTABLE DRUG DELIVERY COMPOSITIONS COMPRISING NON-POLYMERIC SORPTION ENHANCERS AND METHODS OF TREATMENT THEREOF” filed on Mar. 14, 2013, and U.S. Provisional Application No. 61/815,366, entitled “IMPLANTABLE DRUG DELIVERY COMPOSITIONS COMPRISING NON-POLYMERIC SORPTION ENHANCERS AND METHODS OF TREATMENT THEREOF” filed on Apr. 24, 2013; the contents of both which are incorporated by reference.

### FIELD OF THE INVENTION

The invention relates to reservoir-based drug delivery compositions that are implantable into a subject in order to deliver therapeutically effective amounts of a drug, for example, at a pseudo-zero order rate, for extended periods of time (e.g., at least one month, one year, etc.). In particular, the invention relates to the use of non-polymeric acids, bases, and salts for controlling the release rate of a drug from a subcutaneous implant.

### BACKGROUND OF THE INVENTION

Drug compositions come in many different forms and may be administered to a patient via several different routes, such as oral, parenteral, topical, intravenous, subcutaneous, intranasal, etc. Depending on the active and the treatment desired, different routes of administration may be preferable.

Some diseases and conditions may be long lasting, requiring treatment for many weeks, months, or even years. Typically, a patient taking a traditional oral dosage form (e.g., tablets or capsules) may be required to take the oral dose at least once per day for the duration of the treatment. For example, a patient may need to take an oral dose twice a day for a year or longer. One of the problems with treatments that require continuous dosage over a long period of time is that often the patient may not be compliant in taking the medications. In other words, the patient may forget, believe the treatment is unnecessary, or grow tired of having to take many pills over an extremely long period of time. Accordingly, treatments are necessary which can alleviate these compliance issues, but still provide effective and efficient treatment to the patient.

As one example, compliance with breast cancer medications has been an issue. Breast cancer is the leading life-threatening cancer affecting women, and 200,000 new cases of breast cancer are diagnosed each year. Typical treatment is interventional (e.g., chemotherapy/radiation/surgery) followed by adjunctive therapy (e.g., used with or after the primary treatment), where appropriate. After interventional therapy, almost all (e.g., about 95%) hormone receptor positive, post-menopausal patients are prescribed an aromatase inhibitor to suppress estrogen and prevent recurrence. Studies have documented low compliance, low adherence, and low persistence, for example, with patients prescribed a five-year aromatase inhibitor therapy.

As another example, compliance with schizophrenia medications has also been an issue. Schizophrenia is a complex mental disorder, which affects both men and women equally. Although there is no cure for schizophrenia, the treatment success rate with antipsychotic medications and psycho-social therapies can be high. However, an estimated 40% of all relapses suffered by schizophrenic patients are due to noncompliance in taking their prescribed medicine. Patient relapse from noncompliance may also result in the return of more severe and dangerous psychotic symptoms, and persistent noncompliance can worsen the prognosis and make the patient less likely to respond to medication.

Additional examples of diseases or conditions that typically have treatment regimens lasting several years include Parkinson's disease, spasticity, and osteoporosis. Parkinson's disease is a progressive neurodegenerative disorder that is characterized by a patient's selective loss of dopaminergic neurons, which results in motor impairments, such as bradykinesia (i.e., slowness of movement), tremors, muscular rigidity, and postural instability. Spasticity is an involuntary tension, stiffening or contractions of muscles, which typically results from an injury to a part of the central nervous system (e.g., brain or spinal cord) that controls voluntary movements and results in increased activity or excitability in muscles. Spasticity is most often related to cerebral palsy, multiple sclerosis (MS), physical trauma (e.g., a brain or spinal cord injury), a blockage or bleeding in the brain (e.g., a stroke), or an infection (e.g., meningitis or encephalitis). Osteoporosis is a condition in which the bones become weak and break easily, particularly in post-menopausal women.

Accordingly, there has remained a need for effective dosage forms that provide therapeutically effective amounts of a drug over a long period of time, particularly where compliance is an issue, long-term treatment is needed, and/or a steady dose (e.g., zero order) is desired.

### SUMMARY OF THE INVENTION

Aspects of the present invention include reservoir-based drug delivery compositions comprising non-polymeric sorption enhancers, which may be implanted into a subject in order to deliver a therapeutically effective amount of a drug to the subject for long periods of time (e.g., at least one month, at least three months, at least six months, at least one year, at least two years, etc.). The therapeutically effective amount of the drug may be delivered at a pseudo-zero order rate. Accordingly, the present invention is directed to drug compositions, methods of treatment, methods of delivering the drug, subcutaneous delivery systems, and kits regarding the same.

According to an embodiment of the present invention, a drug delivery composition comprises a drug elution rate-controlling excipient comprising an elastomeric polymer defining a reservoir. The reservoir contains at least one discrete solid dosage form comprising at least one active pharmaceutical ingredient (API) and one or more non-polymeric sorption enhancers. The drug delivery composition is in an implantable dosage form.

According to another embodiment of the present invention, a method of delivering a therapeutically effective amount of an active pharmaceutical ingredient (API) from an implantable drug delivery composition comprises implanting a reservoir-based drug delivery composition into a subject to systemically deliver a therapeutically effective amount of the API to the subject at a pseudo-zero order rate for a period of time of at least one month. The drug delivery composition comprises at least one discrete solid dosage form surrounded by an excipient comprising an elastomeric polymer, and the at least one discrete solid dosage form comprises the API and one or more non-polymeric sorption enhancers.

According to another embodiment of the present invention, a method of treating or preventing a disease or condition in a subject comprises implanting a reservoir-based drug delivery composition into a subject to systemically deliver an API to the subject for a period of time of at least one month at a pseudo-zero order elution rate. The drug delivery composition comprises at least one discrete solid dosage form surrounded by an excipient comprising an elastomeric polymer. The at least one discrete solid dosage form comprises the API and one or more non-polymeric sorption enhancers. The drug delivery composition is therapeutically effective to treat or prevent the disease or condition.

According to another embodiment of the present invention, a subcutaneous delivery system comprises a thermoplastic reservoir implant comprising at least one discrete solid dosage form surrounded by a polymeric rate-controlling excipient. The at least one discrete solid dosage form comprises at least one active pharmaceutical ingredient (API) and one or more non-polymeric sorption enhancers. The subcutaneous delivery system provides for release of the API at an elution rate suitable to provide a therapeutically effective amount of the API to a subject at a pseudo-zero order rate for a period of time of at least one month.

According to another embodiment of the present invention, a kit for subcutaneously placing a drug delivery composition comprises a reservoir-based drug delivery composition comprising a rate-controlling excipient defining a reservoir containing at least one discrete solid dosage form. The at least one discrete solid dosage form comprises an API and one or more non-polymeric sorption enhancers. The kit further comprises an implanter for inserting the reservoir-based drug delivery composition beneath the skin.

### BRIEF DESCRIPTION OF THE DRAWINGS

The invention may be further understood by reference to the drawings in which:

FIG. 1 depicts the role of the excipient in a reservoir-based drug delivery composition according to one aspect of the present invention;

FIG. 2 depicts the cylindrical shape of a reservoir-based drug delivery composition according to one embodiment of the present invention;

FIG. 3 depicts the difference between a drug reservoir and a matrix-based implant;

FIG. 4 is a graph showing the elution rate (micro g/day) of risperidone from implants containing glutamic acid monosodium salt, according to embodiments of the present invention described in Example 2;

FIG. 5 is a graph showing the elution rate (micro g/day) of risperidone from implants containing sodium gluconate, according to embodiments of the present invention described in Example 3;

FIG. 6 is a graph showing the elution rate (micro g/day) of risperidone from implants containing EDTA, potassium sulfate, or citric acid, according to embodiments of the present invention described in Example 4;

FIG. 7 is a graph showing the elution rate (micro g/day) of risperidone from implants containing sodium acetate, sodium ascorbate, sodium borate, or sodium citrate, according to embodiments of the present invention described in Example 5; and

FIG. 8 is a graph showing the elution rate (micro g/day) of risperidone from implants containing arginine or tromethamine, according to embodiments of the present invention described in Example 6.

### DETAILED DESCRIPTION OF THE INVENTION

Aspects of the present invention include reservoir-based drug delivery compositions comprising non-polymeric sorption enhancers which may be implanted into a subject in order to deliver a therapeutically effective amount of a drug to the subject for a long period of time (e.g., at least one month, at least three months, at least six months, at least one year, at least two years, etc.). Additional aspects of the present invention include methods of treatment, methods of delivering the drug, subcutaneous delivery systems, and kits regarding the same.

As discussed in more detail below, sorption enhancers are compounds which may improve the release of an API from implantable drug delivery compositions. Previously, sorption enhancers have been polymeric in nature, such as croscarmellose, polyacrylate, PVP and polyethylene glycols. As described in more detail below, the applicants have surprisingly discovered that simple, low molecular weight acids and bases, and salts thereof, can be used as sorption enhancers to help control the release rates of drugs from subcutaneous implants.

As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest, delay the onset of or partially prevent a target disease or condition or one or more symptoms thereof.

The terms “active pharmaceutical ingredient,” “API,” “drug,” or “active” may be used herein interchangeably to refer to the pharmaceutically active compounds) in the drug delivery composition. This is in contrast to other ingredients in the drug delivery composition, such as excipients, which are substantially or completely pharmaceutically inert. A suitable API in accordance with the present invention is one where there is or likely may be patient compliance issues for treating a certain disease or condition, where long-term treatment is needed, and/or where a steady dose (e.g., zero order) is desired.

The term “pharmaceutically acceptable,” as used herein, means approved by a regulatory agency, e.g. of the U.S. Federal or a state government or listed in the U.S. Pharmacopeia or other generally recognized pharmacopeia for use in animals, and more particularly in humans.

The terms “subject” and “patient”, are used interchangeably herein and refer to a mammalian individual, such as a human being.

Each compound used herein may be discussed interchangeably with respect to its chemical formula, chemical name, abbreviation, etc. For example, PTMO may be used interchangeably with poly(tetramethylene oxide). Additionally, each polymer described herein, unless designated otherwise, includes homopolymers, copolymers, terpolymers, and the like.

As used herein and in the claims, the terms “comprising” and “including” are inclusive or open-ended and do not exclude additional unrecited elements, compositional components, or method steps. Accordingly, the terms “comprising” and “including” encompass the more restrictive terms “consisting essentially of” and “consisting of.” Unless specified otherwise, all values provided herein include up to and including the endpoints given, and the values of the constituents or components of the compositions are expressed in weight percent of each ingredient in the composition.

Reservoir-Based Drug Delivery Composition

The drug delivery composition is a reservoir-based drug delivery composition. As used herein, the “reservoir-based composition” is intended to encompass a composition having a substantially or completely closed, surrounded, or encased hollow space or reservoir, where the hollow space or reservoir is filled, at least partially, with at least one discrete solid dosage form.

In one embodiment of the present invention, a drug delivery composition comprises a drug elution rate-controlling excipient comprising an elastomeric polymer defining a reservoir, and the reservoir contains at least one discrete solid dosage form comprising at least one API and one or more non-polymeric sorption enhancers (e.g., one or more non-polymeric acids, bases, or salts). The elastomeric polymer defining the reservoir is formed separate from the at least one discrete solid dosage form (i.e., the elastomeric polymer defining the reservoir and the at least one discrete solid dosage form are not two “layers” that are bonded to each other; rather, the elastomeric polymer defining the reservoir is separately formed and the at least one discrete solid dosage form is placed into contact with the elastomeric polymer when it is loaded into the reservoir).

A reservoir-based composition, as used herein, is in contradistinction to a matrix-based composition. As depicted in FIG. 3, a drug reservoir includes a reservoir portion 120 and a rate controlling portion (excipient 110) whereas a matrix-based implant only consists of the matrix material 130 with the drug incorporated therein. In other words, in a reservoir system, the drug is contained within or is surrounded by some type of rate-controlling material (e.g., a wall, membrane, or casing). In a matrix system, the drug is combined within some type of matrix, often polymeric, which often erodes or degrades in order to release the active to the subject. A matrix system will typically not contain a separate rate-controlling layer because it relies on the chemical composition of the matrix itself to control the rate of drug release.

Thus, there are some major distinctions between the two types of systems. The reservoir-based system allows for a much higher drug loading (e.g., on the order of 98% maximum) whereas a matrix-based system contains a much smaller amount (e.g., on the order of 25% maximum). Although a higher drug loading may be beneficial, it can also be dangerous because of the increased risk of drug overdose or dumping into the subject if the surrounding material were to break or rupture. Additionally, the reservoir-based composition of the present invention allows for a zero-order or pseudo-zero order rate of release of the active. A matrix-based system, on the contrary, typically provides for a first order rate of release when used over long periods of time. A first order rate may be characterized by a high initial rate of release that decays or diminishes quickly over time.

As used herein, the term “pseudo-zero order” or “pseudo-zero order rate” refers to a zero-order, near-zero order, substantially zero order, or controlled or sustained release of an API. A zero order release profile may be characterized by release of a constant amount of the API per unit time. A pseudo-zero order release profile may be characterized by approximating a zero-order release by release of a relatively constant amount of the API per unit time (e.g., within 40%, 30%, 20%, or 10% of the average value). Under a pseudo-zero order rate, the composition may initially release an amount of the API that produces the desired therapeutic effect, and gradually and continually release other amounts of the API to maintain the level of therapeutic effect over an extended period of time (e.g., at least one month, six months, or one year). In order to maintain a near-constant level of API in the body, the API may be released from the composition at a rate that will replace the amount of API being metabolized and/or excreted from the body. It will be appreciated by one of ordinary skill in the art that there may be some initial period of time before steady state is reached (e.g., a ramp up before the target range is reached), which still complies with the definition of “pseudo-zero order.”

Without wishing to be bound to a particular theory, it is believed that a concentration gradient occurs where the concentration of API within the reservoir is “infinite” (e.g., the reservoir acts an infinite supply, but the concentration is practically limited by the amount of active for the given duration of release) and the concentration outside the drug delivery composition is zero (e.g., the subject acts as an infinite sink where the active is constantly being taken away from the composition by the subject's body, such as circulatory, lymphatic systems, etc.). Additionally, the excipient 110 (e.g., the wall through which the active passes) becomes fully saturated with the active ingredient at steady state. Accordingly, this gradient allows the “infinite” supply of API to be adsorbed into the excipient, dissolve in and diffuse through the polymer wall, and then be desorbed for release into the subject. The selection of the excipient 110 may help to provide the pseudo-zero order release of the drug. Without wishing to be limited, it is believed that the release of the drug is not dependent on the desorption from the excipient.

Dosage Form(s)

The drug delivery composition of the present invention comprises at least one dosage form comprising at least one API and one or more non-polymeric sorption enhancers (e.g., one or more non-polymeric acids, bases, or salts).

As used herein, the term “discrete solid dosage form” is intended to encompass any dosage form that is in the form of a solid. The solid dosage form may include any cohesive solid form (e.g., compressed formulations, pellets, tablets, etc.) The solid dosage form may include a solid body or mass comprising the API, which may be prepared in any suitable manner known to one of ordinary skill in the art (e.g., compressed, pelleted, extruded).

The solid dosage forms are “discrete” in that there are one or more dosage forms contained within the reservoir. In other words, the discrete solid dosage form includes one or more solid formulations which are separate and distinct from the polymeric rate-controlling excipient. In an exemplary embodiment, the discrete solid dosage form(s) do not fill the entire reservoir or cavity (e.g., the solid dosage forms are substantially spherical and the reservoir is substantially cylindrical). For example, the solid dosage form need not be co-extruded with the surrounding excipient such that the solid dosage form fills the entire cavity.

The discrete solid dosage forms may be of any suitable shape and of any suitable quantity. In one embodiment of the present invention, the discrete solid dosage forms are substantially spherical in shape. The discrete solid dosage form(s) may be “substantially spherical” in that the solid dosage forms are spherical or nearly spherical in that the length of the longest radius is approximately equal to the shortest radius of the dosage form. For example, the shape of the dosage form may not deviate from a perfect sphere by more than about 10%. In another embodiment, the discrete solid dosage forms comprise more than one pellet (e.g., 2-9 pellets). The number of discrete solid dosage forms may be proportional to the elution rate. In other words, a higher number of dosage forms may result in a higher average elution rate than a smaller number of dosage forms. Thus, it may be preferable to include more discrete solid dosage forms to give a higher elution rate (e.g., 7-9 pellets).

The discrete solid dosage form comprises one or more active pharmaceutical ingredients. A suitable API in accordance with the present invention is one where compliance is at issue, where long term treatment is needed, and/or where a steady dose (e.g., zero order) is required, for example, to minimize side effects. The amount of API is not particularly limited, but may be preferably on the order of about 75-97 wt % of the solid dosage form, or about 80-95 wt % of the solid dosage form.

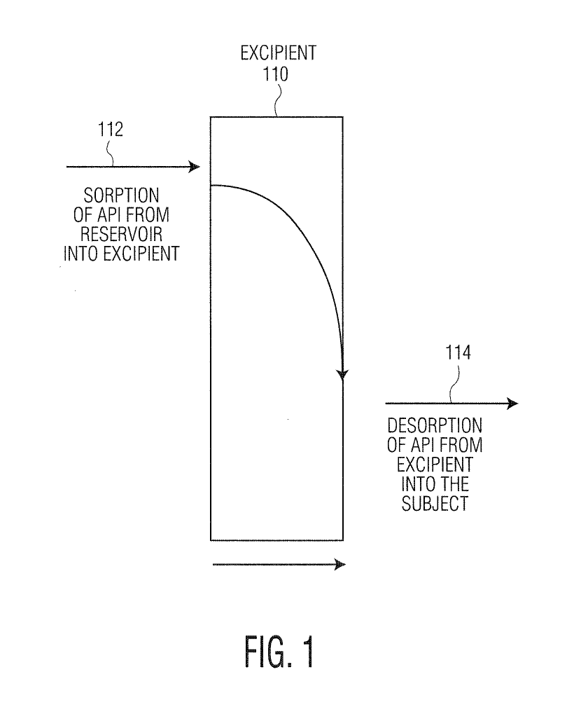

The discrete solid dosage form also comprises one or more non-polymeric sorption enhancers. As used herein, the term “sorption enhancer” is intended to encompass compounds which improve release of the API from the drug delivery composition. Without wishing to be bound to a particular theory, the sorption enhancers may improve release of the API from the drug delivery composition by drawing water or other fluids into the reservoir from the subject, disintegrating or breaking apart the discrete solid dosage form(s), and/or allowing the API to come into contact or remain in contact the inner walls of the excipient. Such a mechanism may be depicted, for example, in FIG. 1. FIG. 1 represents the rate-controlling excipient 110. The API, located in the reservoir on the left side of the diagram, is sorbed 112 from the reservoir to the excipient. The API then crosses through the excipient 110. The API is then desorbed 114 from the excipient into the subject.

Previously, sorption enhancers used in subcutaneous implants have been polymeric in nature. Such “polymeric sorption enhancers” have included, for example, negatively-charged polymers, such as croscarmellose sodium, sodium carboxymethyl starch, sodium starch glycolate, sodium acrylic acid derivatives (e.g., sodium polyacrylate), cross-linked polyacrylic acid (e.g., CARBOPOL®), chondroitin sulfate, poly-glutamic acid, poly-aspartic acid, sodium carboxymethyl cellulose, neutral polymers, such as polyethylene glycol, polyethylene oxide, and polyvinylpyrrolidone. However, the applicants have surprisingly discovered that simple, low molecular weight acids and bases, and salts thereof, can be used as sorption enhancers to help control the release rates of drugs from subcutaneous implants. According to particular embodiments, API's barely eluted (i.e., eluted at very low levels) from drug delivery compositions of the present invention when a sorption enhancer was not included as a component of the discrete solid dosage form(s). However, when one or more non-polymeric sorption enhancers were added to the discrete solid dosage form(s), the API's unexpectedly eluted from the drug delivery compositions at pseudo-zero order elution rates.

As used herein, “non-polymeric sorption enhancers” of the present invention include non-polymeric acids, bases, and salts. According to particular embodiments, non-polymeric sorption enhancers include amino acids and salts thereof (e.g., arginine and salts thereof, glutamic acid and salts thereof, such as glutamic acid monosodium salt, etc.); citric acid and salts thereof (e.g., sodium citrate); salts of tartaric acid, gluconic acid, acetic acid, ascorbic acid, and/or boric acid; and/or polyamino carboxylic acids and salts thereof (e.g., ethylenediaminetetraacetic acid (EDTA)). According to one embodiment, the one or more non-polymeric sorption enhancers are selected from the group consisting of glutamic acid monosodium salt, sodium gluconate, ethylenediaminetetraacetic acid (EDTA), potassium sulfate, citric acid, sodium acetate, sodium ascorbate, sodium borate, sodium citrate, arginine, tromethamine, sodium bitartrate, and combinations thereof.

According to particular embodiments, the one or more non-polymeric sorption enhancers comprise or consist of one or more polyamino carboxylic acids or salts thereof, such as ethylenediaminetetraacetic acid (EDTA). In these embodiments, the API may comprise, for example, paliperidone or raloxifene.

According to other embodiments, the one or more non-polymeric sorption enhancers comprise or consist of one or more salts of ascorbic acid, such as sodium ascorbate. In these embodiments, the API may comprise, for example, paliperidone or raloxifene.

According to other embodiments, the one or more non-polymeric sorption enhancers include ascorbic acid and analogs or derivatives thereof (e.g., erythorbic acid, dehydroascorbic acid, calcium ascorbate dihydrate, ascorbyl palmitate, sodium ascorbyl phosphate, etc.); and/or gallic acid and analogs or derivatives thereof (e.g., n-octyl gallate, propyl gallate, etc.). According to one embodiment, the one or more non-polymeric sorption enhancers are selected from the group consisting of ascorbic acid, erythorbic acid, dehydroascorbic acid, calcium ascorbate dihydrate, ascorbyl palmitate, sodium ascorbyl phosphate, gallic acid, n-octyl gallate, propyl gallate, and combinations thereof.

The average molecular weights of the non-polymeric sorption enhancers used in embodiments of the present invention are not particularly limited, but are preferably less than about 400 g/mol, or less than about 300 g/mol. According to particular embodiments, the average molecular weights of the non-polymeric sorption enhancers range from about 50 g/mol to about 400 g/mol, from about 70 g/mol to about 350 g/mol, or from about 100 g/mol to about 300 g/mol.

According to alternative embodiments, suitable sorption enhancers may include one or more “sugar-based sorption enhancers.” Sugar-based sorption enhancers include monosaccharides (e.g., glucose, fructose, galactose, glucosamine, galactosamine, etc.), disaccharides (e.g., sucrose, lactose, maltose, etc.), and sugar alcohols (preferably formed from monosaccharides or disaccharides, e.g., sorbitol, mannitol, etc.). The sugar-based sorption enhancers may have an acyclic or cyclic structure, and may optionally have one or more modifying groups attached thereto (e.g., carbonyl groups, methyl groups, acyl groups, etc.). According to particular embodiments, sugar-based sorption enhancers may also include oligosaccharides (i.e., sugars having from three to ten monosaccharide units bonded together).

According to particular embodiments, the sugar-based sorption enhancers are selected from the group consisting of monosaccharides, disaccharides, oligosaccharides, sugar alcohols, and combinations thereof. For example, the one or more sugar-based sorption enhancers may be selected from the group consisting of monosaccharides, disaccharides, sugar alcohols formed from monosaccharides or disaccharides, and combinations thereof. According to one embodiment, the one or more sugar-based sorption enhancers are selected from the group consisting of glucosamine, sucrose, lactose, sorbitol, and combinations thereof. For example, the one or more sugar-based sorption enhancers may comprise or consist of glucosamine.

The amount of the non-polymeric sorption enhancer(s) is not particularly limited, but may be present on the order of less than 30 wt % of the solid dosage form, about 1-25 wt % of the solid dosage form, about 2-20 wt % of the solid dosage form, about 4-16 wt % of the solid dosage form, or about 8-12 wt % of the solid dosage form.

The amount of non-polymeric sorption enhancer may be proportional to the elution rate. In other words, a higher weight percent of non-polymeric sorption enhancer in the drug composition may result in a higher average elution rate than a smaller weight percentage. Thus, it may be preferable to include a higher weight percent of non-polymeric sorption enhancer to give a higher elution rate (e.g., 7-25 wt %).

In one embodiment, a suitable sorption enhancer can comprise a combination of two or more non-polymeric and/or sugar-based and/or polymeric sorption enhancers.

The discrete solid dosage form may also comprise other ingredients as long as they do not adversely impact the elution rate. Other suitable ingredients may include, for example, lubricants, excipients, preservatives, etc. A lubricant may be used in the pelleting or tableting process to form the discrete solid dosage form(s), as would be well known by one of ordinary skill in the art. Suitable lubricants may include, but are not limited to, magnesium stearate, calcium stearate, zinc stearate, stearic acid, polyethylene glycol, and the like. The amount of any additional ingredients is not particularly limited, but is preferably on the order of less than about 5 wt % of the solid dosage form, and most preferably less than about 3 wt % of the solid dosage form, particularly preferably about 2% or less of the solid dosage form.

In one embodiment of the present invention, the at least one discrete solid dosage form comprises: 75-97 wt % API based on the total weight of the at least one discrete solid dosage form; 1-25 wt % of at least one non-polymeric sorption enhancer based on the total weight of the at least one discrete solid dosage form; and 0-5 wt % lubricant based on the total weight of the at least one discrete solid dosage form.

Excipient

The discrete solid dosage form(s) is/are surrounded by an excipient. In other words, the discrete solid dosage form(s) is/are substantially or completely surrounded, encased, or enclosed by the excipient. In the present invention, there are no holes or pores in the excipient to allow egress of the API or ingress of bodily fluids, unlike an osmotic system, which requires a hole to allow release of the API. Moreover, there is no (or negligible) build up of pressure within a drug delivery composition in accordance with the present invention, unlike an osmotic system, which requires pressure to force the API out of the device.

In one embodiment of the present invention, the excipient is substantially or completely non-porous. “Substantially nonporous” may refer to a material which has a porosity or void percentage less than about 10%, about 5%, or about 1%, for example. In particular, the excipient is substantially non-porous in that there are no physical pores or macropores, which would allow for egress of the API from the drug delivery composition. In another embodiment, the excipient is practically insoluble in water. Solubility is the concentration of a solute when the solvent has dissolved all the solute that it can at a given temperature (e.g., the concentration of solute in a saturated solution at equilibrium). As used herein, the term “practically insoluble in water” is consistent with the definition in The United States Pharmacopeia—National Formulary (USP-NF) definition, which provides for more than 10,000 parts solvent to one part solute (e.g., one gram of the excipient in greater than 10,000 mL of water).

Without wishing to be bound to a particular theory, it is believed that a concentration gradient across the excipient (e.g., wall, membrane, layer) allows for continuous release of the API. As depicted in FIG. 1, sorption 112 of the API occurs from the reservoir onto the rate-controlling excipient 110. The API then dissolves into and fully saturates the excipient 110, diffuses through it, and the API is then desorbed 114 from the excipient into the subject. Accordingly, this gradient allows the “infinite” supply of API to be adsorbed onto the excipient, diffuse through it and desorbed into the subject, which, based on the excipient selected, may help to provide the pseudo-zero order release of the drug. Thus, the excipient may also be called a drug elution rate-controlling or rate-controlling excipient herein. The “rate-controlling excipient” is intended to encompass materials which control the elution rate of the API. In other words, a polymeric excipient, that when encasing the drug delivery composition, provides a different rate of release, namely, a controlled rate of release (e.g., pseudo-zero order) as compared to the release of an API from an identical composition without a rate-controlling excipient.

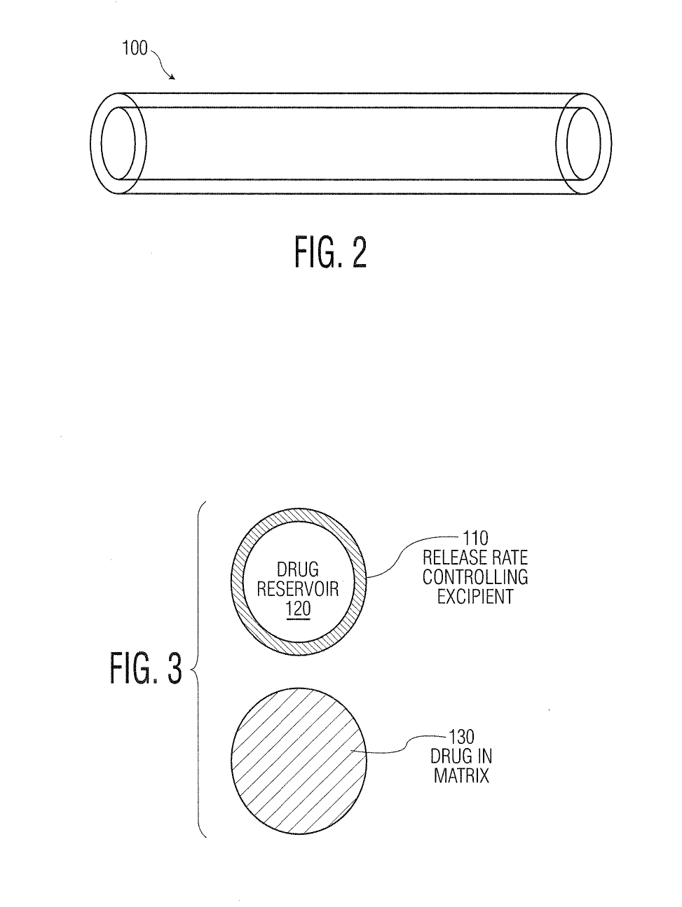

The excipient defines the shape of the reservoir. The reservoir may be of any suitable size and shape. In an exemplary embodiment, the excipient is substantially cylindrically shaped. As used herein, the terms “cylindrical” or “cylindrically shaped” may be used interchangeably to mean at least substantially having the shape of a cylinder. As used herein, the term “cylinder” includes and refers to, but is not limited to: circular cylinders, having a circular cross-section; elliptical cylinders, having an elliptical cross-section; generalized cylinders, having any shape in cross-section; oblique cylinders, in which the end surfaces are not parallel to one another and/or are not normal to the axis of the cylinder; and conical and frusto-conical analogs thereof. In accordance with one aspect of the invention, a hollow tube may include a substantially consistent cross-sectional area and two substantially equally-sized circular ends. The cylindrical shape defines the shape of the excipient defining the reservoir (e.g., the outer portion of the drug delivery composition). An embodiment of the cylindrically shaped excipient is depicted, for example, in FIG. 2. Preferably, the dimensions of the cylindrical hollow tube should be as precise as possible (e.g., a consistent shape and dimension along the length of the tube, in particular, a consistent circular cross-section). The reservoir may be of any suitable size depending on the active and location of delivery. For example, the composition may range in size from about 2 mm to about 4 mm in diameter (e.g., about 2.7 mm in diameter) and about 6 mm to about 50 mm in length, for example about 45 mm in length.

The excipient comprises at least one polymer. Any suitable polymer may be selected by one of ordinary skill in the art, as long as the polymer allows for delivery of a therapeutically effective amount of the API to the subject, for example, at a pseudo-zero order rate, for the intended period of time that the implant resides in a patient. In one embodiment, the polymer comprises a thermoplastic elastomer. As used herein, “thermoplastic,” “thermoplastic elastomers (TPE)” or “thermoplastic rubbers” may be used to denote a class of copolymers or a physical mix of polymers (e.g., a plastic and a rubber), which consist of materials with both thermoplastic and elastomeric properties. The crosslinking in thermoplastic elastomeric polymers may include a weaker dipole or hydrogen bond or the crosslinking occurs in one of the phases of the material. The class of copolymer may include, for example, styrenic block copolymers, polyolefin blends, elastomeric alloys, thermoplastic polyurethanes, thermoplastic copolyester, and thermoplastic polyamides.

As used herein, “elastomer” or “elastomeric polymer” is intended to encompass polymers (homopolymers, copolymers, terpolymers, oligomers, and mixtures thereof) having elastomeric properties (e.g., the tendency to revert to its original shape after extension). In other words, the polymeric backbone may contain one or more elastomeric subunits (e.g., an elastomeric soft segment or block). In one embodiment, the elastomeric polymer comprises polyurethane, polyether, polyamide, polycarbonate, polysilicone, or copolymers thereof. Thus, the elastomeric polymer may include polyurethane-based polymers, polyether-based polymers, polysilicone-based polymers, polycarbonate-based polymers, or combinations thereof.

The polymer may be formed by any suitable means or techniques known to one of ordinary skill in the art. For example, the polymer may be formed from monomers, polymer precursors, pre-polymers, polymers, etc. Polymer precursors may include monomeric as well as oligomeric substances capable of being reacted or cured to form polymers. The polymers may be synthesized using any suitable constituents.

In one embodiment of the present invention, the polymer comprises polyurethanes (e.g., comprising a urethane linkage, —RNHCOOR′—). Polyurethanes may include polyether-based polyurethanes, polycarbonate-based polyurethanes, polyamide-based polyurethanes, polysilicone-based polyurethanes, or the like. Polyurethanes may be formed, for example, from polyols (e.g., comprising two or more hydroxyl or alcohol functional groups, —OH), isocyanates (e.g., comprising an isocyanate group, —N═C═O), and, optional chain extenders, catalysts, and other additives.

Suitable polyols may include, for example, polyether polyols, polycarbonate-based polyols, and the like, which may include diols, triols, etc. Polyether polyols may include, for example, polyalkylene glycols (e.g., polyethylene glycols, polypropylene glycols, polybutylene glycols), poly(ethylene oxide) polyols (e.g., polyoxyethylene diols and triols), polyoxypropylene diols and triols, and the like. Alternative polyols may include, for example, 1,4-butanediol, 1,6-hexanediol, 1,12-dodecanediol, and the like.

For example, the polyol segment or segments may be represented by one or more of the following formulas:

O—(CH₂—CH₂—CH₂—CH₂)ₓ—O— (Formula 1)

—[O—(CH₂)ₙ]ₓ—O— (Formula 2)

O—[(CH₂)₆—CO₃]ₙ—(CH₂)—O— (Formula 3)

Formula (1) may depict a suitable polyether-based polyol, which may be representative of a polyol to produce TECOFLEX® polyurethanes. Formula (2) may depict a suitable polyether-based polyol, which may representative of a polyol to produce TECOPHILIC® polyurethanes. Formula (3) may depict a suitable polycarbonate-based polyol, which may be representative of a polyol to produce CARBOTHANE® polyurethanes (all of which are obtainable from the Lubrizol Corporation with offices in Wickliffe, Ohio). The polyols may also include mixtures of one or more types of polyol segments.

Suitable isocyanates may include, for example, aliphatic and cycloaliphatic isocyanates, such as 1,6-hexamethylene diisocyanate (HDI), 1-isocyanato-3-isocyanatomethyl-3,5,5-trimethyl-cyclohexane (isophorone diisocyanate, IPDI), and 4,4′-diisocyanato dicyclohexylmethane (H12MDI).

Suitable chain extenders may include, for example, ethylene glycol, 1,4-butanediol (1,4-BDO or BDO), 1,6-hexanediol, cyclohexane dimethanol, and hydroquinone bis(2-hydroxyethyl) ether (HQEE).

In one embodiment of the present invention, the polymer comprises a polyether-based polyurethane. For example, the polymer may be an aliphatic polyether-based polyurethane comprising poly(tetramethylene oxide) and polymerized 4,4′-diisocyanato dicyclohexylmethane (H12MDI) and 1,4-butanediol. An exemplary type of suitable polyether-based polyurethanes includes TECOFLEX® polymers available from the Lubrizol Corporation. For example, TECOFLEX® polymers include aliphatic block copolymer with a hard segment consisting of polymerized 4,4′-diisocyanato dicyclohexylmethane (H12MDI) and 1,4-butanediol, and a soft segment consisting of the macrodiol poly(tetramethylene oxide). In one embodiment, the TECOFLEX® polymer comprises TECOFLEX® EG-93A polyurethane. In another embodiment, the TECOFLEX® polymer comprises TECOFLEX® EG-80A polyurethane.

In another embodiment of the present invention, the polymer comprises polyether-amides (e.g., thermoplastic poly(ether-block-amide)s, e.g., PEBA, PEB, TPE-A, and commercially known as PEBAX® polyether-amides obtainable from Arkema Chemicals Inc., headquartered in Philadelphia, Pa.). Synthesis may be carried out, for example, in the molten state by polycondensation between polyether blocks (e.g., a diol, such as polyoxyalkylene glycols) and polyamide blocks (e.g., carboxylic acid terminated amide blocks, such as dicarboxylic blocks), which results in a thermoplastic copolymer. The long chain molecules may consist of numerous blocks where the polyamide provides rigidity and the polyether provides flexibility to the polymer. Thus, the polyether-amides may consist of linear chains of hard polyamide (PA) blocks covalently linked to soft polyether (PE) blocks via ester groups. The polyether-amides may also be synthesized via a catalyst (e.g., metallic Ti(OR)₄), which facilitates the melt polycondensation of the polyether and polyamide blocks. The general structural formula of these block copolymers may be depicted as follows:

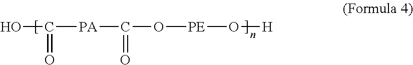

The polyamide block may include various amides including nylons (such as nylon 6, nylon 11, nylon 12, etc.). The polyether block may also include various polyethers, such as polytetramethylene oxide (PTMO), polypropylene oxide (PPO), polyethylene glycol (PEG), poly(hexamethylene oxide), polyethylene oxide (PEO), and the like. The ratio of polyether to polyamide blocks may vary from 80:20 to 20:80 (PE:PA). As the amount of polyether increases, a more flexible, softer material may result.

For example, the thermoplastic elastomer may be selected from the group consisting of TECOFLEX® polyurethanes, CARBOTHANE® polyurethanes, PEBAX® polyether-amides, and combinations thereof. For example, the elastomer may include TECOFLEX® EG-93A polyurethane, TECOFLEX® EG-80A polyurethane, TECOFLEX® EG-85A polyurethane, PEBAX® 2533 polyether-amide, PEBAX® 3533 polyether-amide, CARBOTHANE® PC-3585A polyurethane, and combinations thereof.

TECOFLEX® polyurethanes and CARBOTHANE® polyurethanes are described, for example, in Lubrizol's brochure for Engineered Polymers for Medical & Healthcare dated 2011, the disclosure of which is hereby incorporated by reference in its entirety, for all purposes. For example, TECOFLEX® aliphatic polyether polyurethanes may have the following characteristics:

TABLE 1

| Product | Hardness | Flex Modulus | Feature |
|---|---|---|---|
| EG80A | 72A | 1,000 | Clear |
| EG85A | 77A | 2,300 | Clear |
| EG93A | 87A | 3,200 | Clear |
| EG100A | 94A | 10,000 | Clear |
| EG60D | 51D | 13,000 | Clear |
| EG65D | 60D | 37,000 | Clear |
| EG68D | 63D | 46,000 | Clear |
| EG72D | 67D | 92,000 | Clear |
| EG80A B20/B40 | 73A/78A | 1,200/1,500 | Radiopaque |
| EG85A B20/B40 | 83A/86A | 2,700/3,700 | Radiopaque |
| EG93A B20/B40 | 90A/95A | 5,000/4,700 | Radiopaque |
| EG100A B20/B40 | 93A/98A | 17,000/14,000 | Radiopaque |
| EG60D B20/B40 | 55D/65D | 27,000/27,000 | Radiopaque |
| EG65D B20/B40 | 63D/78D | 82,000/97,000 | Radiopaque |
| EG68D B20 | 73D | 76,600 | Radiopaque |
| EG72D B20/B40 | 75D/82D | 125,000/179,000 | Radiopaque |

CARBOTHANE® aliphatic polycarbonate polyurethanes may have the following characteristics, for example:

TABLE 2

| Product | Hardness | Flex Modulus | Feature |
|---|---|---|---|
| PC-3575A | 71A | 620 | Clear |
| PC-3585A | 78A | 1,500 | Clear |
| PC-3595A | 91A | 4,500 | Clear |
| PC-3555D | 52D | 24,000 | Clear |
| PC-3572D | 71D | 92,000 | Clear |
| PC-3575A-B20 | 79A | 860 | Radiopaque |
| PC-3585A-B20 | 81A | 1,700 | Radiopaque |
| PC-3595A-B20 | 90A | 8,600 | Radiopaque |
| PC-3555D-B20 | 54D | 25,000 | Radiopaque |
| PC-3572D-B20 | TBD | 141,000 | Radiopaque |

According to alternative embodiments, a drug delivery composition of the present invention comprises a polymeric excipient that comprises or consists of a thermoplastic, aromatic polyurethane (e.g., the polyurethane comprises aromatic diisocyanates, such as methylene diphenyl diisocyanate (MDI)). The reservoir contains at least one discrete solid dosage form comprising at least one active pharmaceutical ingredient (API), and the drug delivery composition is in an implantable dosage form. Non-limiting examples of suitable aromatic diisocyanates include methylene diphenyl diisocyanate (MDI), toluene diisocyanate (TDI), p-phenylene diisocyanate (PPDI), naphthalene diisocyanate (NDI), and combinations thereof.

According to one embodiment, the aromatic polyurethane comprises poly(tetramethylene oxide) (PTMO) and an aromatic diisocyanate. The PTMO preferably has a molecular weight between about 500 daltons to about 5,000 daltons (e.g., between about 1,500 daltons to about 3,500 daltons, or between about 2,000 daltons to about 3,000 daltons). In one embodiment of the present invention, for example, the polymer is an aromatic polyurethane comprising poly(tetramethylene oxide) (PTMO) and polymerized methylene diphenyl diisocyanate (MDI) and 1,4-butanediol.

The polymers may be processed using any suitable techniques, such as extrusion, injection molding, compression molding, spin-casting. For example, the polymer may be extruded or injection molded to produce hollow tubes having two open ends (see e.g., FIG. 2). The hollow tube can be loaded with the discrete solid dosage form(s). The open ends are sealed to form the reservoir-based drug delivery composition. A first open end may be sealed before filling the tube with the discrete solid dosage form(s), and the second open end may be sealed after the tube is filled with all of the discrete solid dosage form(s). The tube may be sealed using any suitable means or techniques known in the art. For example, the ends may be plugged, filled with additional polymers, heat sealed, or the like. The tubes should be permanently sealed such that the discrete solid dosage form(s) may not be removed. Also, the ends should be suitably sealed such that there are no holes or openings that would allow egress of the active once implanted.

The wall thickness of the excipient may be selected to provide for the desired elution rate. The wall thickness may be inversely proportional to elution rate. Thus, a larger wall thickness may result in a lower elution rate. The excipient may form a wall having an average thickness of about 0.05 to about 0.5 mm, or about 0.1 mm to about 0.3 mm (e.g., about 0.1 mm, about 0.2 mm, or about 0.3 mm).

In one embodiment of the present invention, the drug delivery composition does not require erosion or degradation of the excipient in vivo in order to release the API in a therapeutically effective amount. Alternatively, the excipient is not substantially erodible and/or not substantially degradable in vivo for the intended life of the implantable composition. As used herein, “erosion” or “erodible” are used interchangeably to mean capable of being degraded, disassembled, and/or digested, e.g., by action of a biological environment. A compound that is “not substantially erodible” is not substantially degraded, disassembled, and/or digested over time (e.g., for the life of the implant). Alternatively, the material may be “not substantially erodible” or “does not require erosion” in vivo in order to provide for release of the API. In other words, the compound may erode over time, but the API is not substantially released due to erosion of the material. With respect to “degradation” or “degradable,” these are intended to mean capable of partially or completely dissolving or decomposing, e.g., in living tissue, such as human tissue. Degradable compounds can be degraded by any mechanism, such as hydrolysis, catalysis, and enzymatic action. Accordingly, a compound that is “not substantially degradable” does not substantially dissolve or decompose over time (e.g., for the life of the implant) in vivo. Alternatively, the material may be “not substantially degradable” or “not requiring degradation” in order to provide for release of the API. In other words, the compound may degrade over time, but the API is not substantially released due to degradation of the material.

Methods of Treatment

Embodiments of the present invention include methods of treating or preventing one or more diseases or conditions comprising implanting a reservoir-based drug delivery composition into a subject to deliver a therapeutically effective amount of an API to the subject for long periods of time (e.g., at least one month, at least three months, at least six months, at least one year, etc.). Suitable active pharmaceutical ingredients in accordance with the present invention may include active pharmaceutical ingredients in oral dosage forms where compliance is at issue, where long term treatment is needed, and/or where a steady dose (e.g., zero order) is required, for example, to minimize side effects. In other words, suitable APIs may be selected for the treatment of diseases and conditions that are long-lasting (e.g., requiring treatment for many weeks, months or even years). Diseases and conditions may include, but are not limited to, estrogen related disorders (e.g., breast cancer, short stature in children or adolescents), psychotic disorders (e.g., schizophrenia, Bipolar disorder), benign prostatic hyperplasia, overactive bladder, Parkinson's disease, smoking cessation, Alzheimer's, Sickle cell anemia, pulmonary arterial hypertension, autoimmune diseases (e.g., multiple sclerosis, Crohn's disease, Lupus), spasticity, osteoporosis, restless legs syndrome, pain, itch, interstitial cystitis, overactive bladder, and the like. The methods of treatment described herein may treat, delay onset, or inhibit recurrence of the disease or condition. A pharmaceutically effective or therapeutic amount of API should be administered sufficient to affect or produce the desired therapy.

Treatment of Estrogen-Related Disorders

Treatment of an estrogen-related disorder or estrogen-receptor disorder may include treatment of any estrogen-related or estrogen-receptor disorders, diseases, or conditions known to one of ordinary skill in the art, such as breast cancer, endometriosis, uterine fibroids (also called leiomyomas), short stature in children or adolescents and the like. Estrogen-related disorders may include high estrogen levels or normal estrogen levels that need to be reduced. It is intended that a pharmaceutically effective amount of an API would be administered via the drug delivery composition, which will inhibit, or at least partially arrest or partially prevent or suppress estrogen. For example, treatment may include treatment that can suppress or delay the recurrence of breast cancer. For the treatment of estrogen-related disorders, the active may include aromatase inhibitors, such as anastrozole, letrozole, exemestane, etc.

For the case of aromatase inhibitors as the API, releasing an amount of aromatase inhibitor effective to inhibit or slow onset or recurrence of an estrogen-related disorder, such as breast cancer (e.g., lower estrogen levels), is desired. A doctor would be able to determine the efficacy of the treatment (i.e., know the aromatase inhibitor was working to treat breast cancer) using techniques known to one of ordinary skill in the art. For example, one method for assessing the effectiveness of aromatase inhibitor(s) in the treatment of an estrogen-related disorder, such as breast cancer, comprises measuring mean serum concentrations of estradiol in a subject over time.

In another embodiment of the present invention, the estrogen-related disorder is short stature in children or adolescents. Estrogens have been found to be important for bone maturation, growth plate fusion, and cessation of longitudinal growth in children and adolescents. For the treatment of short stature, the active may include aromatase inhibitors, such as anastrozole, letrozole, etc. According to preferred embodiments, treating short stature in a child or adolescent (e.g., a male child or adolescent) with an aromatase inhibitor is effective in increasing the predicted adult height of the subject. A subject's predicted adult height can be determined by examining the subject's bone age according to known methods. Following treatment with the aromatase inhibitor, the bone age of the subject may be examined again to determine if the predicted adult height had increased, which would indicate that the aromatase inhibitor had been effective in increasing the subject's projected adult height. Another method may comprise monitoring the subject's bone age acceleration before, during, and after treatment with an aromatase inhibitor.

According to an embodiment of the present invention, a method for treating an estrogen-related disorder (e.g., breast cancer, endometriosis, uterine fibroids, short stature in children, etc.) comprises implanting a reservoir-based drug delivery composition into a subject to systemically deliver a therapeutically effective amount of an aromatase inhibitor (e.g., anastrozole, letrozole, etc.) to the subject for a period of time of at least one month (e.g., at a pseudo-zero order elution rate), wherein the drug delivery composition comprises at least one discrete solid dosage form surrounded by an excipient comprising an elastomeric polymer. The at least one discrete solid dosage form comprises the aromatase inhibitor or a pharmaceutically acceptable salt thereof and one or more non-polymeric sorption enhancers. The drug delivery composition is therapeutically effective to treat the estrogen-related disorder.

Treatment of the Symptoms of a Psychotic Disorder

The treatment of the symptoms of a psychotic disorder (such as schizophrenia, bipolar disorder, or autism) can require long lasting treatment, often on the order of many years, even for the life of the patient. Compliance with antipsychotic medications has also been an ongoing issue. The treatment of a psychotic disorder in accordance with the present invention may be directed to men or women. By “treatment,” it is intended that a pharmaceutically effective amount of risperidone would be administered via the drug delivery composition, which will cure, prevent, inhibit, or at least partially arrest or partially prevent or suppress the symptoms of the psychotic disorder. The treatment is particularly effective in that once the implant is administered to the patient, the patient will continue to receive a therapeutically effective dose for the intended duration of the implant (e.g., one year). This is in contrast to the oral dosage form, which requires compliance by the patient and continued oral administration consistently over the same duration of time.

As used herein, the terms “psychotic disorder” or “psychosis” may be used interchangeably to refer to a disorder in which psychosis is a recognized symptom. The symptoms of psychosis may include, but are not limited to, hallucinations, delusions, paranoia, mania, depression, emotional changes, personality changes, behavioral changes, and lack of awareness of mental changes. The term “antipsychotic” refers to drugs used to treat psychosis. Common conditions for which antipsychotics are prescribed include schizophrenia, mania, and delusional disorders. Antipsychotics also act as mood stabilizers making them suitable for the treatment of bipolar disorder (even when no symptoms of psychosis are present).

The psychotic disorder may include, for example, schizophrenia, bipolar disorder, autism, or any variations thereof. Schizophrenia may include various types including paranoid subtype, disorganized subtype, catatonic subtype, undifferentiated subtype, or residual subtype. For example, the paranoid subtype (also known as paranoid schizophrenia) may include the presence of auditory hallucinations or prominent delusional thoughts about persecution or conspiracy. Bipolar disorder may include different versions including bipolar disorder I, bipolar disorder II, cyclothymic disorder, bipolar disorder not otherwise specified (NOS), or bipolar disorder with rapid cycling. For example, bipolar disorder I may be characterized by at least one manic episode or mixed episode (symptoms of both mania and depression occurring simultaneously), and one or more depressive episodes, that lasts for at least 7 days. Autism includes developmental disorders that appear in the first 3 years of life and affects the brain's normal development of social and communication skills.

For the case of risperidone, releasing an amount of risperidone effective to inhibit, stabilize or slow onset or recurrence of psychotic conditions, such as schizophrenia, or symptoms thereof is desired. A doctor would be able to determine the efficacy of the treatment (i.e., know the risperidone was working to treat the psychotic disorder) using techniques known to one of ordinary skill in the art. For example, after a subject has begun a regimen of risperidone, a clinician may use a rating scale which assesses the psychiatric symptoms of schizophrenia, for example, in order to determine whether there has been an improvement in those symptoms over time. The Brief Psychiatric Rating Scale (BPRS) is a multi-item inventory of general psychopathology which may be used to evaluate the effects of risperidone treatment, for example, in a schizophrenia patient. The BPRS psychosis cluster (conceptual disorganization, hallucinatory behavior, suspiciousness, and unusual thought content) is considered a particularly useful subset for assessing schizophrenic patients. Another traditional assessment, the Clinical Global Impression (CGI), reflects the impression of a skilled observer that is fully familiar with the manifestations of the psychotic disorder (e.g., schizophrenia), about the overall clinical state of the patient. In addition, the Positive and Negative Syndrome Scale (PANSS) and the Scale for Assessing Negative Symptoms (SANS) may be employed. Improvement in a subject's symptoms, as measured by a clinician according to any of the aforementioned assessments, or other assessments used in the art to evaluate the symptoms of a psychotic disorder, can be used to indicate whether the amount of risperidone being used is effective.

In one embodiment of the present invention, a method of treating the symptoms of a psychotic disorder (e.g., schizophrenia, bipolar disorder, or autism) comprises implanting a reservoir-based drug delivery composition into a subject to systemically deliver a therapeutically effective amount of risperidone or a pharmaceutically acceptable salt thereof to the subject for a period of time of at least one month (e.g., at a pseudo-zero order elution rate). The drug delivery composition comprises at least one discrete solid dosage form surrounded by an excipient comprising an elastomeric polymer, the at least one discrete solid dosage form comprising risperidone or a pharmaceutically acceptable salt thereof and one or more non-polymeric sorption enhancers. The drug delivery composition is therapeutically effective to treat the symptoms of the psychotic disorder.

Treatment of Parkinson's Disease

Monoamine oxidase B (MAO-B) inhibitors, such as rasagiline, provide an alternative first-line treatment for the symptoms of Parkinson's disease, or serve as an adjunctive treatment in addition to other drugs, such as levodopa. One mechanism of action of rasagiline is believed to be its MAO-B inhibitory activity, which causes an increase in extracellular levels of dopamine in the brain. Treatment of one or more of the symptoms of Parkinson's disease according to embodiments of the present invention include treatment of one or more symptoms known to one of ordinary skill in the art. Symptoms of Parkinson's disease may include, but are not limited to, motor impairments such as bradykinesia (i.e., slowness of movement), problems with balance, muscular rigidity, postural instability, and/or tremors. Symptoms of Parkinson's disease may also include, but are not limited to, non-motor symptoms, such as bladder and bowel dysfunction, postural hypotension, anxiety, apathy, dementia, depression, psychosis, pain, and/or sleep disturbances.

The treatment of one or more of the symptoms of Parkinson's disease can require long-lasting treatment, often on the order of many years. The treatment of synnptom(s) of Parkinson's disease in accordance with the present invention is directed to early or advanced Parkinson's disease, and to monotherapy (i.e., as a subject's only dopaminergic medication) or adjunctive therapy (i.e., used in addition to (with or after) treatment with one or more other dopaminergic medications, typically levodopa).

By “treatment,” it is intended that a pharmaceutically effective amount of rasagiline would be administered via the drug delivery composition, which will inhibit, or at least partially arrest or partially prevent or suppress one or more symptoms of Parkinson's disease. For example, treatment may include treatment that can suppress one or more motor impairments, such as bradykinesia, muscular rigidity, postural instability, and/or tremors. The treatment is particularly effective in that once the implant is administered to the patient, the patient will continue to receive a therapeutically effective dose for the intended duration of the implant (e.g., one month, three months, six months, one year, 18 months, two years, 30 months, or more).

The methods of treatment described herein may treat, delay onset, suppress, or inhibit one or more symptoms of Parkinson's disease. A pharmaceutically effective or therapeutic amount of rasagiline should be administered sufficient to effect or produce the desired therapy. For example, releasing an amount of rasagiline effective to inhibit or suppress one or more symptoms of Parkinson's disease (e.g., bradykinesia, tremors, muscular rigidity, and/or postural instability) is desired. A doctor would be able to determine the efficacy of the treatment (i.e., know the rasagiline was working to treat symptoms of Parkinson's disease) using techniques known to one of ordinary skill in the art. For example, after a subject has begun a regimen of rasagiline, a clinician may use a rating scale which assesses the symptoms of Parkinson's disease in order to determine whether there has been an improvement in those symptoms over time. One measure of effectiveness is the Unified Parkinson's Disease Rating Scale (UPDRS). Improvement in a subject's symptoms, as measured by a clinician according to the aforementioned assessment, or other assessments used in the art to evaluate the symptoms of Parkinson's disease, can be used to indicate whether the amount of rasagiline being used is effective.

According to one aspect of the present invention, a method of treating one or more symptoms of Parkinson's disease comprises implanting a reservoir-based drug delivery composition into a subject to systemically deliver a therapeutically effective amount of rasagiline to the subject for a period of time of at least one month (e.g., at a pseudo-zero order elution rate). The drug delivery composition comprises at least one discrete solid dosage form surrounded by an excipient comprising an elastomeric polymer, the at least one discrete solid dosage form comprising rasagiline or a pharmaceutically acceptable salt thereof (e.g., rasagiline hemitartrate) and one or more non-polymeric sorption enhancers. The drug delivery composition is therapeutically effective to treat the one or more symptoms of Parkinson's disease. For example, rasagiline may be administered to treat depression in a subject with Parkinson's disease.

Treatment of Spasticity

Spasticity is an involuntary tension, stiffening or contractions of muscles, which typically results from an injury to a part of the central nervous system (e.g., brain or spinal cord) that controls voluntary movements and results in increased activity or excitability in muscles. Spasticity is most often related to cerebral palsy, multiple sclerosis (MS), physical trauma (e.g., a brain or spinal cord injury), a blockage or bleeding in the brain (e.g., a stroke), or an infection (e.g., meningitis or encephalitis). Symptoms of spasticity, e.g., the involuntary tension, stiffening or contractions of muscles, may range from slight muscle stiffness to permanent shortening of the muscle (contracture). Additional symptoms of spasticity may include, but are not limited to, increased muscle tone, overactive reflexes, involuntary movements that may include spasms (brisk and/or sustained involuntary muscle contractions) or clonus (a series of fast involuntary contractions), pain, decreased functional abilities and delayed motor development, abnormal posture, and contractures (permanent contractions of the muscle and tendon due to severe persistent stiffness and spasms). Spasticity may be constantly present or event-triggered, and may result in pain that impacts daily life activities.

Tizanidine is an imidazoline central α₂-adrenoceptor agonist that is effective at managing spasticity. Tizanidine is in a class of medications called skeletal muscle relaxants, and works by slowing action in the brain and nervous system to allow muscles to relax. Treatment of one or more of the symptoms of spasticity according to embodiments of the present invention include treatment of one or more symptoms known to one of ordinary skill in the art, such as those discussed above.

By “treatment,” it is intended that a pharmaceutically effective amount of tizanidine would be administered via the drug delivery composition, which will inhibit, or at least partially arrest or partially prevent or suppress one or more symptoms of spasticity. For example, treatment may include treatment that can suppress involuntary tension, stiffening and/or contractions of muscles. The treatment is particularly effective in that once the implant is administered to the patient, the patient will continue to receive a therapeutically effective dose for the intended duration of the implant (e.g., one month, three months, six months, one year, 18 months, two years, 30 months, or more).

The methods of treatment described herein may treat, delay onset, suppress, or inhibit one or more symptoms of spasticity. A pharmaceutically effective or therapeutic amount of tizanidine should be administered sufficient to effect or produce the desired therapy. For example, releasing an amount of tizanidine effective to inhibit or suppress one or more symptoms of spasticity (e.g., involuntary tension, stiffening or contraction of muscles) is desired. A doctor would be able to determine the efficacy of the treatment (i.e., know the tizanidine was working to treat symptoms of spasticity) using techniques known to one of ordinary skill in the art. For example, after a subject has begun a regimen of tizanidine, a clinician may conduct a clinical examination to assess strength and reflexes, using rating scales such as the Ashworth Scale or Modified Ashworth Scale (which provide an objective score of muscle tone based on range of motion). Alternatively, the clinician may make functional measurements using assessments such as the Fugl-Meyer Assessment, which provides an objective score based on motor functioning, balance, sensation and joint functioning. Improvement in a subject's symptoms, as measured by a clinician according to the aforementioned assessments, or other assessments used in the art to evaluate the symptoms of spasticity, can be used to indicate whether the amount of tizanidine being used is effective.

According to one aspect of the present invention, a method of treating one or more symptoms of spasticity comprises implanting a reservoir-based drug delivery composition into a subject to systemically deliver a therapeutically effective amount of tizanidine to the subject for a period of time of at least one month (e.g., at a pseudo-zero order elution rate). The drug delivery composition comprises at least one discrete solid dosage form surrounded by an excipient comprising an elastomeric polymer, the at least one discrete solid dosage form comprising tizanidine or a pharmaceutically acceptable salt thereof (e.g., tizanidine free base) and one or more non-polymeric sorption enhancers. The drug delivery composition is therapeutically effective to treat the one or more symptoms of spasticity.

Treatment and Prevention of Osteoporosis and Invasive Breast Cancer

According to embodiments of the present invention, the treatment or prevention of an estrogen-related disorder may include the treatment or prevention of osteoporosis (e.g., in a post-menopausal woman), or decreasing the risk of invasive breast cancer (e.g., in a post-menopausal woman, such as a post-menopausal woman with osteoporosis or a post-menopausal woman with a high risk of developing invasive breast cancer). The binding of raloxifene to estrogen receptors results in the activation of certain estrogenic pathways and the blockade of others. Thus, raloxifene is an estrogen agonist/antagonist.

Raloxifene is currently indicated for the prevention and treatment of osteoporosis in post-menopausal women. Osteoporosis is a condition in which the bones become thin and weak and break easily. By “treatment,” it is intended that a pharmaceutically effective amount of raloxifene would be administered via a drug delivery composition of the present invention, which will reverse or stop the progression of osteoporosis, or which will inhibit, or at least partially arrest or partially prevent or suppress the progression of osteoporosis. By “prevention,” it is intended that a pharmaceutically effective amount of raloxifene would be administered via a drug delivery composition of the present invention, which will prevent, inhibit, or at least partially arrest or partially prevent or suppress the development of osteoporosis in a subject that has not yet developed or shown signs of osteoporosis.

A doctor would be able to determine the efficacy of the treatment (i.e., know the raloxifene was working to produce the desired therapy) using techniques known to one of ordinary skill in the art. For example, after a subject has begun a regimen of raloxifene to treat osteoporosis, a clinician may conduct a clinical examination to assess reductions in the subject's serum or urine levels of bone turnover markers (e.g., bone-specific alkaline phosphatase, osteocalcin, or collagen breakdown products), decreases in bone resorption based on radiocalcium kinetics studies, increases in bone mineral density (BMD), and/or decreases in the incidence of fractures. A clinician may alternatively use conventional radiography to assess bone density. Improvement in a subject's symptoms, as measured by a clinician according to the aforementioned assessments, or other assessments used in the art to evaluate osteoporosis, can be used to indicate whether the amount of raloxifene being used is effective.

Raloxifene is also indicated for decreasing the risk of developing invasive breast cancer (i.e., breast cancer that has spread outside of the milk ducts or lobules into surrounding breast tissue) in post-menopausal women who are at a high risk of developing invasive breast cancer, or in post-menopausal women who have osteoporosis. A patient may have a high risk of breast cancer if she has had at least one abnormal breast biopsy (e.g., a biopsy showing lobular carcinoma in situ or atypical hyperplasia), one or more first-degree relatives (e.g., a mother, sister, or daughter) with breast cancer, or a 5-year predicted risk of breast cancer 1.66% (based on the modified Gail model).

According to an embodiment of the present invention, a method for treating or preventing an estrogen-related disorder in a subject comprises decreasing the risk of breast cancer (e.g., invasive breast cancer) from developing in the subject. In particular embodiments, the subject is a post-menopausal woman, such as a post-menopausal woman with osteoporosis or a post-menopausal woman with a high risk of developing invasive breast cancer. By “decreasing the risk” of invasive breast cancer from developing in a subject, it is intended that a pharmaceutically effective amount of raloxifene would be administered via a drug delivery composition of the present invention, which will prevent, inhibit, or at least partially arrest or partially prevent or suppress the development of invasive breast cancer in a subject that has not developed invasive breast cancer.

According to one aspect of the present invention, a method for treating or preventing osteoporosis, and/or decreasing the risk of invasive breast cancer, comprises implanting a reservoir-based drug delivery composition into a subject (e.g., a post-menopausal woman) to systemically deliver a therapeutically effective amount of raloxifene to the subject for a period of time of at least one month (e.g., at a pseudo-zero order elution rate). The drug delivery composition comprises at least one discrete solid dosage form surrounded by an excipient comprising an elastomeric polymer, the at least one discrete solid dosage form comprising raloxifene or a pharmaceutically acceptable salt thereof (e.g., raloxifene free base) and one or more non-polymeric sorption enhancers. The drug delivery composition is therapeutically effective to treat or prevent osteoporosis and/or decrease the risk of invasive breast cancer.

Treatment of Neurological Disorders

According to embodiments of the present invention, a method for treating one or more symptoms of a neurological disorder in a subject comprises treating one or more symptoms of Parkinson's disease in the subject (e.g., idiopathic Parkinson's disease). In another embodiment, a method for treating one or more symptoms of a neurological disorder in a subject comprises treating one or more symptoms of RLS in the subject (e.g., moderate-to-severe primary restless legs syndrome). Pramipexole is currently indicated for treating the signs and symptoms of Parkinson's disease (e.g., idiopathic Parkinson's disease). The mechanism of action of pramipexole as a treatment for Parkinson's disease is believed to be related to its ability to stimulate dopamine receptors in the striatum. Pramipexole is also indicated for treating restless legs syndrome (RLS) (e.g., moderate-to-severe primary restless legs syndrome). RLS is a neurological disorder that affects the legs (and sometimes arms or other parts of the body) and causes an uncontrollable urge to move them, especially at night and when sitting or lying down, and is usually accompanied by uncomfortable and sometimes painful sensations in the legs.

A doctor would be able to determine the efficacy of the treatment (i.e., know the pramipexole was working to treat symptoms of Parkinson's disease or RLS) using techniques known to one of ordinary skill in the art. One measure of effectiveness is the Unified Parkinson's Disease Rating Scale (UPDRS). As another example, after a subject has begun a regimen of pramipexole, a clinician may use the International RLS Rating Scale (IRLS Scale), which assesses the symptoms of RLS in order to determine whether there has been an improvement in symptoms over time.

According to one aspect of the present invention, a method for treating one or more symptoms of Parkinson's disease or restless legs syndrome comprises implanting a reservoir-based drug delivery composition into a subject to systemically deliver a therapeutically effective amount of pramipexole to the subject for a period of time of at least one month (e.g., at a pseudo-zero order elution rate). The drug delivery composition comprises at least one discrete solid dosage form surrounded by an excipient comprising an elastomeric polymer, the at least one discrete solid dosage form comprising pramipexole or a pharmaceutically acceptable salt thereof (e.g., pramipexole free base) and one or more non-polymeric sorption enhancers. The drug delivery composition is therapeutically effective to treat the one or more symptoms of Parkinson's disease or restless legs syndrome.

Treatment of Pain, Itch, Interstitial Cystitis and Overactive Bladder

The methods, compositions, and kits of the invention can also be used to treat pain, itch, interstitial cystitis and/or overactive bladder resulting from a number of conditions. Lidocaine is a synthetic amide that is well-known for its sedative, analgesic, and cardiac depressant properties. The term “pain” as used herein includes all types of pain. In one embodiment, the pain may be acute or chronic. In another embodiment, the pain may be nociceptive, dysfunctional, idiopathic, neuropathic, somatic, visceral, inflammatory, and/or procedural. The term “itch” refers to all types of itching and stinging sensations that may be localized or generalized, and may be acute, intermittent or persistent. The itch may be idiopathic, allergic, metabolic, infectious, drug-induced, or due to specific disease states due to liver or kidney disease, or cancer.

By “treatment,” it is intended that a pharmaceutically effective amount of lidocaine would be administered via a drug delivery composition of the present invention, which will partially or fully suppress, arrest, inhibit, or prevent pain, itch, interstitial cystitis and/or overactive bladder. In one embodiment, the pain, itch, interstitial cystitis or overactive bladder may be eliminated permanently or for a short period of time. In another embodiment, the severity of the pain, itch, interstitial cystitis or overactive bladder may be lessened permanently, or for a short period of time.

According to one aspect of the present invention, a method for treating pain, itch, interstitial cystitis and/or overactive bladder comprises implanting a reservoir-based drug delivery composition into a subject to systemically or locally deliver a therapeutically effective amount of lidocaine to the subject for a period of time of at least one month (e.g., at a pseudo-zero order elution rate). The drug delivery composition comprises at least one discrete solid dosage form surrounded by an excipient comprising an elastomeric polymer, the at least one discrete solid dosage form comprising lidocaine or a pharmaceutically acceptable salt thereof (e.g., lidocaine free base) and one or more non-polymeric sorption enhancers. The drug delivery composition is therapeutically effective to treat pain, itch, interstitial cystitis and/or overactive bladder.

Active Pharmaceutical Ingredients

As discussed above, suitable active pharmaceutical ingredients in accordance with the present invention may include active pharmaceutical ingredients in oral dosage forms where compliance is at issue, where long term treatment is needed, and/or where a steady dose (e.g., zero order) is required, for example, to minimize side effects. In other words, suitable APIs may be selected for the treatment of diseases and conditions that are long-lasting (e.g., requiring treatment for many weeks, months or even years). For example, the active pharmaceutical ingredient may be selected from the group consisting of anastrozole, exemestane, dutasteride, oxybutynin, letrozole, selegiline, tolterodine, tizanidine, varenicline, rivastigmine, rasagiline, asenapine, paliperidone, aripiprazole, rotigotine, folic acid, vardenafil, fingolimod, laquinimod, risperidone, nicergoline, guanfacine, raloxifene, pramipexole, lidocaine, histrelin, and pharmaceutically acceptable salts thereof (e.g., HCl, tartrate, mesylate, maleate, palmitate, and the like). According to particular embodiments, the API has a molecular weight that is less than or equal to about 8,000 g/mol, less than or equal to about 4,000 g/mol, less than or equal to about 2,000 g/mol, or less than or equal to about 1,000 g/mol. In one embodiment of the present invention, the selected API is hydrophobic.

For the treatment of estrogen-related disorders, the active may include aromatase inhibitors, such as anastrozole, letrozole, exemestane, etc. For the treatment of psychotic disorders including schizophrenia, the active may include risperidone, asenapine (e.g., asenapine maleate), paliperidone, etc. For the treatment of Bipolar disorder, the active may include risperidone, aripiprazole, etc. For the treatment of benign prostatic hyperplasia, the active may include dutasteride, etc. For the treatment of overactive bladder, the active may include oxybutynin (e.g., oxybutynin HCl or oxybutynin free base), tolterodine (e.g., tolterodine tartrate), etc. For the treatment of Parkinson's disease, the active may include monoamine oxidase B inhibitors, such as selegiline (e.g., selegiline HCl), rasagiline (e.g., rasagiline hemitartrate), rotigotine, pramipexole (e.g., pramipexole free base), etc. For the treatment of spasticity, the active may include tizanidine (e.g., tizanidine free base), etc. For smoking cessation, the active may include varenicline (e.g., varenicline free base), etc. For the treatment of Alzheimer's, the active may include rivastigmine (e.g., rivastigmine tartrate), etc. For the treatment of sickle cell anemia, the active may include folic acid, etc. For the treatment of pulmonary arterial hypertension, the active may include vardenafil, etc. For the treatment of autoimmune diseases including multiple sclerosis, Crohn's disease, or Lupus, the active may include fingolimod (e.g., fingolimod free base), laquinimod, etc. For the treatment or prevention of osteoporosis or invasive breast cancer, the active may include raloxifene (e.g., raloxifene free base). For the treatment of pain, itch, interstitial cystitis, or overactive bladder the active may include lidocaine (e.g., lidocaine free base), etc. For the treatment of hormone-related diseases or conditions, such as prostate cancer in men, uterine fibroids in women, or central precocious puberty in children, the active may include histrelin (e.g., histrelin acetate), etc. For the treatment of depression, the active may include selegiline, etc.

Implantation

The drug delivery composition may be implanted into the subject in any suitable area of the subject using any suitable means and techniques known to one of ordinary skill in the art. For example, the composition may be implanted subcutaneously, e.g., at the back of the upper arm or the upper back (e.g. in the scapular region). As used herein, the terms “subcutaneous” or “subcutaneously” or “subcutaneous delivery” means directly depositing in or underneath the skin, a subcutaneous fat layer, or intramuscularly. The drug delivery composition may be delivered subcutaneously using any suitable equipment or techniques. In one embodiment, the drug delivery composition is placed subcutaneously in the subject's arm. Alternative sites of subcutaneous administration may also be used as long as a pharmaceutically acceptable amount of the API would be released into the subject in accordance with the present invention. Preferably, the drug delivery composition should not migrate significantly from the site of implantation. Methods for implanting or otherwise positioning the compositions into the body are well known in the art. Removal and/or replacement may also be accomplished using suitable tools and methods known in the art.

Once implanted, the reservoir-based drug delivery composition may systemically deliver a therapeutically effective amount of the API to the subject at a pseudo-zero order rate for a long duration (e.g., a period of time of at least one month). As used herein, the term “systemic” or “systemically” refers to the introduction of the API into the circulatory, vascular and/or lymphatic system (e.g., the entire body). This is in contrast to a localized treatment where the treatment would only be provided to a specific, limited, localized area within the body. Thus, the API may be systemically delivered to the subject by implanting the drug delivery composition subcutaneously into the subject.

According to embodiments of the present invention in which the reservoir-based drug delivery composition comprises lidocaine as the API, the lidocaine may be delivered locally to a specific, limited, or localized area within the body. For example, the drug delivery composition may be subcutaneously implanted in or near an area of the subject's body where there is localized pain or itch, or in or near the bladder of subjects suffering from interstitial cystitis or overactive bladder. The drug delivery composition may deliver lidocaine locally to the site of pain, itch, interstitial cystitis, or overactive bladder, while also delivering lidocaine systemically.

A therapeutically effective amount of the API is delivered to the subject at a pseudo-zero order rate. Pseudo-zero order refers to a zero-order, near-zero order, substantially zero order, or controlled or sustained release of the API. A pseudo-zero order release profile may be characterized by approximating a zero-order release by release of a relatively constant amount of the API per unit time (e.g., within about 30% of the average value). Thus, the composition may initially release an amount of the API that produces the desired therapeutic effect, and gradually and continually release other amounts of the API to maintain the level of therapeutic effect over the intended duration (e.g., about one year). In order to maintain a near-constant level of API in the body, the API may be released from the composition at a rate that will replace the amount of API being metabolized and/or excreted from the body.

Without wishing to be bound to a particular theory, it is believed that the reservoir-based drug composition works by releasing the API through the excipient membrane or wall. In other words, the active diffuses across the excipient, e.g., as depicted in FIG. 1. Thus, sorption 112 of the active occurs from the reservoir onto the rate-controlling excipient 110. The active fully saturates the excipient 110 at steady state, and the active diffuses through the excipient and is then desorbed 114 from the excipient into the subject at a pseudo-zero order rate.

The therapeutically effective amount of the active may be delivered to the subject at a target range between a maximum value and a minimum value of average daily elution rate for the API. As used herein, the term “elution rate” refers to a rate of API delivery, which in one embodiment is based on the oral dose rate multiplied by the fractional oral bioavailability, which may be depicted as follows:

Oral Dose×Fractional Oral Bioavailability %=Target Elution Rate (mg/day)

The elution rate may be an average rate, e.g., based on the mean average for a given period of time, such as a day (i.e., average daily elution rate). Thus, a daily elution rate or average daily elution rate may be expressed as target daily oral dosage multiplied by oral bioavailability. For example, a desired daily dose of 1 mg/day for a drug that has 85% oral bioavailability would expect delivery of about 850 micrograms per day.

The maximum and minimum values refer to a maximum average daily elution rate and a minimum average daily elution rate, respectively. The minimum value required for a pharmaceutically effective dose may be correlated to or determined from a trough value for an oral dosage version of the API (e.g., based on the blood/plasma concentrations for oral formulations). Similarly, maximum value may be correlated to or determined from the peak value for an oral dosage version of the API (e.g., the maximum blood/plasma concentration when an oral dosage is first administered or a pharmaceutically toxic amount). In other words, the target range is a range between maximum and minimum average daily elution rates, respectively, which may be determined based on blood/plasma concentrations for equivalent oral dosage forms containing the same active.

The drug delivery composition is long lasting. In other words, the API is delivered to the subject (e.g., at a pseudo-zero order rate) for an extended period of time. For example, the API is delivered to the subject for at least about one month (about one month or greater), at least about three months (about three months or greater), at least about six months (about six months or greater), at least about one year (about one year or greater), or any period of time within those ranges.

Prior to implantation, the drug delivery composition may undergo any suitable processing, such as sterilization (such as by gamma radiation), heat treatment, molding, and the like. Additionally, the drug delivery composition may be conditioned or primed by techniques known in the art. For example, the drug delivery composition may be place in a medium (e.g., an aqueous medium, such as saline). The medium, priming temperature, and time period of priming can be controlled to optimize release of the active upon implantation.

Subcutaneous Delivery Systems and Kits

In one aspect of the present invention, a subcutaneous delivery system comprises an elastomeric reservoir implant comprising at least one discrete solid dosage form surrounded by a polymeric rate-controlling excipient. The at least one discrete solid dosage form comprises at least one API and one or more non-polymeric sorption enhancers. The subcutaneous delivery system provides for release of the API at an elution rate suitable to provide a therapeutically effective amount of the API to a subject at a pseudo-zero order rate for a period of time of at least one month. In another aspect of the present invention, a kit for subcutaneously placing a drug delivery composition comprises a reservoir-based drug delivery composition comprising a polymeric rate-controlling excipient defining a reservoir containing at least one discrete solid dosage form comprising at least one API and one or more non-polymeric sorption enhancers; and an implanter for inserting the reservoir-based drug delivery composition beneath the skin.

As discussed above, the drug delivery composition may be implanted into the subject in any suitable area of the subject using any suitable means and techniques known to one of ordinary skill in the art. For example, the composition may be implanted subcutaneously, e.g., at the back of the upper arm, by directly depositing in or underneath the skin, a subcutaneous fat layer, or intramuscularly.

The drug delivery composition may be delivered subcutaneously using any suitable equipment or techniques, e.g., an implanter known to one ordinary skill in the art. The kits may comprise the drug delivery composition pre-loaded into the implanter or the drug delivery composition may be loaded by the doctor or other user. The implanter may be an implantation device, such as a syringe, cannula, trocar or catheter, that may be inserted into an incision made at the delivery site of the subject. Suitable implantation devices and implantation methods include the trocar and methods disclosed in U.S. Pat. No. 7,214,206 and U.S. Pat. No. 7,510,549, the disclosures of which are herein incorporated by reference in their entirety, for all purposes. Other suitable methods for implanting or otherwise positioning the compositions into the body, e.g., by a doctor, are well known in the art. Removal and/or replacement may also be accomplished using suitable tools and methods known in the art. Kits may also comprise other equipment well known in the art, such as scalpels, clamps, suturing tools, hydration fluid, and the like.

Implantable Drug Delivery Compositions with Polymer Excipient(s)

Without wishing to be bound to a particular theory, it is believed that by selecting specific polymers with certain contents or ratios of hard to soft segments, certain desired elution rates may be achieved. Moreover, by adding non-polymeric sorption enhancers in certain amounts with the API to the discrete solid dosage formulations within the reservoir, the elution rates may be further changed or modulated (e.g., “tuned” or “dialed in”) from the drug delivery composition to desired, pharmaceutically efficacious values.

According to one aspect of the present invention, a method of delivering a therapeutically effective amount of an active pharmaceutical ingredient from an implantable drug delivery composition comprises implanting a reservoir-based drug delivery composition into a subject to systemically deliver a therapeutically effective amount of an active pharmaceutical ingredient to the subject at a pseudo-zero order rate for a period of time of at least one month. The drug delivery composition comprises at least one discrete solid dosage form surrounded by an excipient comprising at least one polymer, and the at least one discrete solid dosage form comprises the active pharmaceutical ingredient or a pharmaceutically acceptable salt thereof and one or more non-polymeric sorption enhancers. The polymer comprises a substantially non-porous, elastomeric polymer comprising soft and hard segments, and the relative content of the soft and hard segments provide an elution rate within a target range between a maximum and minimum value of a desired average daily elution rate for the active pharmaceutical ingredient.

According to one embodiment of the present invention, a drug delivery composition includes a rate-controlling excipient defining a reservoir which contains at least one discrete solid dosage form comprising an active pharmaceutical ingredient or a pharmaceutically acceptable salt thereof and one or more non-polymeric sorption enhancers. The rate-controlling excipient comprises a substantially non-porous, elastomeric polymer comprising soft and hard segments selected based on the relative content of soft and hard segments of the polymer to obtain an elution rate within a target range of average daily elution rate for the active pharmaceutical ingredient. The at least one discrete solid dosage form comprises at least one non-polymeric sorption enhancer in an amount effective to modulate the average daily elution rate of the active pharmaceutical ingredient to provide for release of the active pharmaceutical ingredient at pseudo-zero order within the target range at the therapeutically effective amount for a period of time of at least one month. The amount of non-polymeric sorption enhancer may be directly proportional to the average daily elution rate. For example, higher amounts of non-polymeric sorption enhancers may result in higher release rates.

According to another aspect of the present invention, two or more sorption enhancers which produce different average daily elution rates in a given implant, if each is used alone, can be combined to achieve a single desired elution rate. For example, a polymeric, non-polymeric or sugar-based sorption enhancer which produces a first average daily elution rate in a given implant when used alone can be combined with a polymeric, non-polymeric or sugar-based sorption enhancer which produces a second average daily elution rate in a given implant when used alone (wherein the second average daily elution rate is less than the first average daily elution rate) to produce an average daily elution rate that is intermediate between the first and second average daily elution rates.

According to another embodiment of the present invention, a method of choosing an implantable drug delivery composition comprises selecting a rate-controlling excipient comprising a substantially non-porous, elastomeric polymer comprising soft and hard segments for defining a reservoir based on the relative content of soft and hard segments of the polymer to adjust the elution rate within a target range of average daily elution rate for an active pharmaceutical ingredient; and selecting and formulating the active pharmaceutical ingredient or a pharmaceutically acceptable salt thereof and at least one non-polymeric sorption enhancer in order to modulate the elution rate to achieve a therapeutically effective amount of the active pharmaceutical ingredient at pseudo-zero order for a period of time of at least one month, wherein the amount of sorption enhancer may be directly proportional to the average daily elution rate.

Polymer Selection

The excipient comprises at least one polymer having soft and hard segments. As used herein, the term “segment” may refer to any portion of the polymer including a monomer unit, or a block of the polymer, or a sequence of the polymer, etc. “Soft segments” may include a soft phase of the polymer, which is amorphous with a glass transition temperature below the use temperature (e.g., rubbery). “Hard segments” may include a hard phase of the polymer that is crystalline at the use temperature or amorphous with a glass transition temperature above the use temperature (e.g., glassy). The use temperature may include a range of temperatures including room temperature (about 20-25° C.) and body temperature (about 37° C.). Without wishing to be bound to a particular theory, the soft segment may provide for the greatest impact on sorption onto the excipient and the hard segment may impact diffusion across or through the excipient. See e.g., FIG. 1 showing sorption 112 of the API from the reservoir into the excipient 110 and desorption 114 of the API from the excipient into the subject. Any suitable polymer comprising hard and soft segments may be selected by one of ordinary skill in the art, as long as the polymer allows for delivery of a therapeutically effective amount of the API to the subject at a pseudo-zero order rate for the intended period of time of the implant. In one embodiment of the present invention, the selected polymer excipient is hydrophobic.

In one embodiment, the polymer is a thermoplastic elastomer or elastomeric polymer, which encompasses polymers (homopolymers, copolymers, terpolymers, oligomers, and mixtures thereof) having elastomeric properties and containing one or more elastomeric subunits (e.g., an elastomeric soft segment or block). The thermoplastic elastomers may include copolymers (e.g., styrenic block copolymers, polyolefin blends, elastomeric alloys, thermoplastic polyurethanes, thermoplastic copolyester, and thermoplastic polyamides) or a physical mix of polymers (e.g., a plastic and a rubber), which consist of materials with both thermoplastic and elastomeric properties, for example, comprising a weaker dipole or hydrogen bond or crosslinking in one of the phases of the material. The elastomeric polymer may comprise polyurethanes, polyethers, polyamides, polycarbonates, polysilicones, or copolymers thereof. Thus, the polymer may include elastomeric polymers comprising polyurethane-based polymers, polyether-based polymers, polysilicone-based polymers, polycarbonate-based polymers, or combinations thereof. In an exemplary embodiment, the polymer comprises a polyurethane-based polymer or a polyether-block-polyamide polymer.

Suitable hard and soft segments of the polymer may be selected by one of ordinary skill in the art. It will be appreciated by one of ordinary skill in the art that although certain types of polymers are described herein, the hard and soft segments may be derived from monomers, polymers, portions of polymers, etc. In other words, the polymers listed may be changed or modified during polymerization, but those polymers or portions of those polymers in polymerized form constitute the hard and soft segments of the final polymer.

Examples of suitable soft segments include, but are not limited to, those derived from (poly)ethers, (poly)carbonates, (poly)silicones, or the like. For example, the soft segments may be derived from alkylene oxide polymers selected from the group consisting of poly(tetramethylene oxide) (PTMO), polyethylene glycol (PEG), poly(propylene oxide) (PPO), poly(hexamethylene oxide), and combinations thereof. The soft segment may also be derived from polycarbonate soft segments (obtainable from Lubrizol) or silicone soft segments (obtainable from Aortech).

Examples of suitable hard segments include, but are not limited to, those derived from polyurethanes or polyamides. For example, the hard segments may be derived from isocyanates and amides, such as nylons, nylon derivatives (such as nylon 6, nylon 11, nylon 12, etc.), carboxylic acid terminated amide blocks, and the like.

The polymer may be formed by any suitable means or techniques known to one of ordinary skill in the art. For example, the polymer may be formed from monomers, polymer precursors, pre-polymers, polymers, etc. Polymer precursors may include monomeric as well as oligomeric substances capable of being reacted or cured to form polymers. The polymers may be synthesized using any suitable constituents.

In one embodiment of the present invention, the polymer comprises polyurethanes (e.g., comprising a urethane linkage, —RNHCOOR′—). Polyurethanes may include polyether-based polyurethanes, polycarbonate-based polyurethanes, polyamide-based polyurethanes, polysilicone-based polyurethanes, or the like, as discussed in detail above.

Polyurethanes may contain both soft segments and hard segments. The soft segments may be derived from polyols including polyether polyols, polycarbonate-based polyols, and the like. For example, soft segments may be derived from polyether polyols, such as polyalkylene glycols (e.g., polyethylene glycols, polypropylene glycols, polybutylene glycols), poly(ethylene oxide) polyols (e.g., polyoxyethylene diols and triols), polyoxypropylene diols and triols, and the like. Soft segments may be derived from polyols, such as 1,4-butanediol, 1,6-hexanediol, 1,12-dodecanediol, and the like. The soft segment derived from the polyols may be represented by the following formulas or mixtures thereof, for example:

O—(CH₂—CH₂—CH₂—CH₂)ₓ—O— (Formula 1)

—[O—(CH₂)ₙ]ₓ—O— (Formula 2)

O—[(CH₂)₆—CO₃]ₙ—(CH₂)—O— (Formula 3)

The hard segments may be derived from isocyanates, such as aliphatic and cycloaliphatic isocyanates, such as 1,6-hexamethylene diisocyanate (HDI), 1-isocyanato-3-isocyanatomethyl-3,5,5-trimethyl-cyclohexane (isophorone diisocyanate, IPDI), and 4,4′-diisocyanato dicyclohexylmethane (H12MDI).

In another embodiment of the present invention, the polymer may comprise a polyether-based polyurethane. For example, the polymer may be an aliphatic polyether-based polyurethane comprising poly(tetramethylene oxide) as the soft segment and polymerized 4,4′-diisocyanato dicyclohexylmethane (H12MDI) and 1,4-butanediol as the hard segment. A suitable polymer includes TECOFLEX®, an aliphatic block copolymer with a hard segment consisting of polymerized 4,4′-diisocyanato dicyclohexylmethane (H12MDI) and 1,4-butanediol, and a soft segment consisting of the macrodiol poly(tetramethylene oxide).

In another embodiment of the present invention, the polymer comprises polyether-amides (e.g., thermoplastic poly(ether-block-amide)s, e.g., PEBA, PEB, TPE-A, and commercially known as PEBAX® polyether-amides). The hard segment may comprise the polyamide blocks (e.g., carboxylic acid terminated amide blocks, such as dicarboxylic blocks) and the soft segments may comprise the polyether blocks (e.g., a diol, such as polyoxyalkylene glycols). The general structural formula of these block copolymers may be depicted as follows:

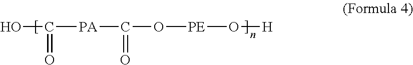

where PA represents the hard segment and PE represents the soft segment. The polyamide block may include various amides including nylons (such as nylon 6, nylon 11, nylon 12, etc.). The polyether block may also include various polyethers, such as poly(tetramethylene oxide) (PTMO), polyethylene glycol (PEG), poly(propylene oxide) (PPO), poly(hexamethylene oxide), polyethylene oxide (PEO), and the like. The ratio of polyether to polyamide blocks may vary from 80:20 to 20:80 (PE:PA). As the amount of polyether increases, a more flexible, softer material may result.

In one embodiment, the elastomeric polymer is selected from the group consisting of TECOFLEX® polyurethanes, CARBOTHANE® polyurethanes, PEBAX® polyether-amides, and combinations thereof. For example, the elastomeric polymer may include TECOFLEX® EG-93A polyurethane, TECOFLEX® EG-80A polyurethane, TECOFLEX® EG-85A polyurethane, PEBAX® 2533 polyether-amide, PEBAX® 3533 polyether-amide, CARBOTHANE® PC-3585A polyurethane, and combinations thereof.

The relative content of the soft and hard segments may provide an elution rate within a target range of average daily elution rate for the active pharmaceutical ingredient. The relative content of the soft and hard segments refers to the amount or content of soft segments to hard segments in the polymer. The relative content may also be defined as a ratio of soft segment to hard segments (e.g., at least about 2:1 or at least about 4:1 of soft to hard segments). For example, the soft content may be 50% or more, 60% or more, 70% or more, or 80% or more relative to the hard content. In one embodiment, the relative content is about 70% soft segments and about 30% hard segments or at least about 2.3:1 soft:hard (e.g., PEBAX® 2533 polyether-amide). In another embodiment, the relative content is about 80% soft segments and about 20% hard segments or at least about 4:1 soft:hard (e.g., PEBAX® 3533 polyether-amide).

The ratio of soft to hard segments may vary depending on the desired elution rate. Without wishing to be bound to a particular theory, it is believed that the soft segments may contribute to the sorption of the API into the excipient and/or the hard segment may contribute to the rate of diffusion (e.g., how fast the active diffuses through the excipient). The rate of diffusion through the excipient probably does not matter much, however, once the implant reaches steady state (e.g., a constant or near constant elution rate). Thus, it may be desirable to have a higher ratio of soft segments relative to hard segments (e.g., at least about 2:1, at least about 3:1, or at least about 4:1). The relative content of the soft and hard segments may also be considered directly proportional on the molecular weights of both the soft and hard segments. In other words, for a given ratio, a higher molecular weight polymer for the soft segment results in a higher relative content of soft segments to hard segments.

The molecular weights of each of the soft and hard segments may be selected depending on the specific soft and hard segments selected. In particular, the size (e.g., molecular weight) of the soft segment may impact the elution rate. For example, the soft block (e.g., polyether) molecular weights may range from about 1000-12,000 daltons (daltons may be used interchangeably with g/mol for molecular weight). For the case of PTMO as the soft segment, the molecular weights may range from about 1000-3000 daltons. In some cases, a higher molecular weight may be preferred (e.g., about 2000-2900 daltons) in order to elevate elution, as compared to less than about 1000 daltons. For the case of PPO as the soft segment, the molecular weight may range from about 2000-12,0000 daltons, and again a higher molecular weight may be preferred to elevate elution rates. For the case of polyether-block amides, the molecular weight of the polyether block may vary from about 400 to about 3000 daltons and that of the polyamide block may vary from about 500 to about 5000 daltons. Without wishing to be bound to a particular theory, it is believed that by increasing the molecular weight of soft segments in the polymer, the content of hard segments is reduced providing for better dissolution and diffusion of the API through the excipient.

The Shore D hardness or Shore hardness of the polymer segments may also have an impact on the elution rates. In some cases, the Shore hardness may be inversely proportional to the elution rate (e.g., a higher Shore hardness results in a lower elution rate). For example, in the case of polyether-block amides, a Shore hardness of 35 provides a lower elution rate as compared to a Shore hardness of 25.

In one embodiment of the present invention, the excipient is substantially or completely non-porous, in that the polymer has a porosity or void percentage less than about 10%, about 5%, or about 1%, for example. In particular, the excipient is substantially non-porous in that there are no physical pores or macropores which would allow for egress of the API from the drug delivery composition. In another embodiment, the excipient is practically insoluble in water, which equates to one gram in >10,000 mL of water. In another embodiment of the present invention, the drug delivery composition does not require erosion or degradation of the excipient in vivo in order to release the API in a therapeutically effective amount. Alternatively, the excipient is not substantially erodible and/or not substantially degradable in vivo for the intended life of the implantable composition (e.g., the API is not released due to erosion or degradation of the material in vivo).

The rate-controlling excipient may comprise a substantially non-porous, elastomeric polymer comprising soft and hard segments selected based on the relative content of soft and hard segments of the polymer to obtain an elution rate within a target range of average daily elution rate for the active pharmaceutical ingredient. A therapeutically effective amount of the API is delivered to the subject at a pseudo-zero order rate within a target range between a maximum and minimum value of a desired average daily elution rate for the active pharmaceutical ingredient. Pseudo-zero order refers to a zero-order, near-zero order, substantially zero order, or controlled or sustained release of the API. The composition may initially release an amount of the API that produces the desired therapeutic effect, and gradually and continually release other amounts of the API to maintain the level of therapeutic effect over the intended duration of treatment (e.g., about one year).

As previously noted, the excipient defines the shape of the reservoir, which may be of any suitable size and shape. In an exemplary embodiment, the excipient is substantially cylindrically shaped. An embodiment of a cylindrically shaped excipient is depicted, for example, in FIG. 2. The reservoir may be of any suitable size depending on the active and location of delivery, e.g., a ratio of about 1:1.5 to 1:5 diameter to length.

The wall thickness of the excipient may also be selected to provide for the desired elution rate. The wall thickness may be inversely proportional to elution rate. Thus, a larger wall thickness may result in a lower elution rate. The excipient may form a wall having an average thickness of about 0.05 to about 0.5 mm, or about 0.1 mm to about 0.3 mm (e.g., about 0.1 mm, about 0.2 mm, or about 0.3 mm).

The polymers may be processed using any suitable techniques, such as extrusion, injection molding, compression molding, spin-casting. In one embodiment, a method of making an implantable drug delivery composition includes: (a) selecting a substantially non-porous elastomeric polymer comprising soft and hard segments based on the relative content and molecular weights of the soft and hard segments of the polymer to provide an elution rate within a target range of average daily elution rate for an active pharmaceutical ingredient; (b) forming a hollow tube from the elastomeric polymer (see e.g., FIG. 2); (c) selecting and formulating the active pharmaceutical ingredient or a pharmaceutically acceptable salt thereof and at least one non-polymeric sorption enhancer in order to produce an elution rate at a therapeutically effective amount of the active pharmaceutical ingredient at pseudo-zero order for a period of time of at least one month, wherein the amount of sorption enhancer is directly proportional to the average daily elution rate; (d) loading at least one discrete solid dosage form comprising the active pharmaceutical ingredient and the at least one non-polymeric sorption enhancer into the tube; and (e) sealing both ends of the tube to form a sealed cylindrical reservoir-based drug delivery composition. The tube may be sealed using any suitable means or techniques known in the art. For example, the ends may be plugged, filled with additional polymers, heat sealed, or the like. The tubes should be permanently sealed such that the discrete solid dosage forms may not be removed. Also, the ends should be suitably sealed such that there are no holes or openings that would allow egress of the active once implanted.

Sorption Enhancer(s) and the Discrete Dosage Form

In another aspect of the present invention, the at least one discrete solid dosage form, within the reservoir, comprises one or more non-polymeric sorption enhancers in an amount effective to modulate the average daily elution rate of the active pharmaceutical ingredient to provide for release of the active pharmaceutical ingredient at pseudo-zero order within the target range at the therapeutically effective amount for a period of time of at least one month. As used herein, the terms “modulate” or “modulation” may be used to describe a change in the activity of the drug delivery composition. This may equate to a change in elution rate (e.g., an increase or a decrease in a given elution rate or range).

As noted above, sorption enhancers may improve the release of the API from the drug delivery composition. Without wishing to be bound to a particular theory, the sorption enhancers may improve release of the API from the drug delivery composition by drawing water or other fluids into the reservoir from the subject, disintegrating or breaking apart the discrete solid dosage form(s), and/or allowing the API to come into contact or remain in contact the inner walls of the excipient. Such a mechanism may be depicted, for example, in FIG. 1.

The amount of the non-polymeric sorption enhancer is not particularly limited, but may be present on the order of less than 30 wt % of the solid dosage form, about 1-25 wt % of the solid dosage form, about 2-20 wt % of the solid dosage form, about 4-16 wt % of the solid dosage form, or about 8-12 wt % of the solid dosage form. The amount of sorption enhancer may be directly proportional to the elution rate. In other words, a higher weight percent of sorption enhancer in the composition may result in a higher average elution rate than a smaller weight percentage. Thus, it may be preferable to include a higher weight percent of sorption enhancer to give a higher elution rate (e.g., about 7-25 wt %). According to particular embodiments, an increase in the concentration of the one or more sorption enhancers provides a higher average elution rate of the API.

In one embodiment of the present invention, the at least one discrete solid dosage form comprises: 75-97 wt % API based on the total weight of the at least one discrete solid dosage form; 1-25 wt % of at least one non-polymeric sorption enhancer based on the total weight of the at least one discrete solid dosage form; and 0-5 wt % lubricant based on the total weight of the at least one discrete solid dosage form. For example, the at least one discrete solid dosage form may comprise: 85-95 wt % API based on the total weight of the at least one discrete solid dosage form; 5-20 wt % of at least one non-polymeric sorption enhancer based on the total weight of the at least one discrete solid dosage form; and 0-5 wt % lubricant (e.g., stearic acid or magnesium stearate) based on the total weight of the at least one discrete solid dosage form. Preferably, each component of the drug delivery composition is provided in an amount effective for the treatment of the disease or condition being treated.

The therapeutically effective amount of the API may be delivered to the subject at a target range of average daily elution rate for the API. The target elution rate (mg/day) is based on the oral dose rate multiplied by the fractional oral bioavailability. The elution rate may be an average rate, e.g., based on the mean average for a given period of time, such as a day (i.e., average daily elution rate). The average daily elution rate of the active pharmaceutical ingredient may vary in direct proportion to the amount of sorption enhancer in the drug delivery composition (e.g., more sorption enhancer may provide for a higher average daily elution rate).

As previously discussed, the minimum value(s) for the average daily elution rate may be correlated to the trough value for an oral dosage version of the API (e.g., based on the blood/plasma concentrations for oral formulations). Similarly, the maximum value(s) may be correlated to the peak value for an oral dosage version of the API (e.g., the maximum blood/plasma concentration when an oral dosage is first administered or a pharmaceutically toxic amount). In other words, the target range is between maximum and minimum elution rates, respectively, which may be determined based on blood/plasma concentrations for equivalent oral dosage forms containing the same active.

The number and shape of the discrete dosage form(s) may be optimized to provide for the desired elution rates. For example, the discrete solid dosage forms may be of suitable shape to not fill the entire cavity of the reservoir. In one embodiment, the at least one discrete dosage form is substantially spherical in shape in that the solid dosage forms are spherical or nearly spherical. For example, the shape of the dosage form may not deviate from a perfect sphere by more than about 10%. The number of discrete dosage forms may be selected to provide a given elution rate. The discrete solid dosage forms may comprise more than one pellet (e.g., 2-9 pellets). The number of discrete solid dosage forms may be directly proportional or related to the elution rate. In other words, a higher number of dosage forms may result in a higher average elution rate than a smaller number of dosage forms. Thus, it may be preferable to include more discrete solid dosage forms to give a higher elution rate (e.g., 7-9 pellets).

Drug Delivery Compositions, Subcutaneous Delivery Systems, and Kits

As previously noted, the drug delivery composition is long lasting, and the API may be delivered to the subject at a pseudo-zero order rate for an extended period of time (e.g., at least about one month (about one month or greater), at least about three months (about three months or greater), at least about six months (about six months or greater), at least about one year (about one year or greater), or any period of time within those ranges).

According to one embodiment of the present invention, a subcutaneous delivery system for releasing an active pharmaceutical ingredient at a pseudo-zero order comprises an elastomeric reservoir implant comprising a rate-controlling excipient defining a reservoir. The rate-controlling excipient comprises a substantially non-porous elastomeric polymer having a relative content of hard segments and soft segments to provide an elution rate within a target range of average daily elution rate for the active pharmaceutical ingredient. The reservoir containing at least one discrete solid dosage form comprising the active pharmaceutical ingredient or a pharmaceutically acceptable salt thereof and an effective amount of at least one non-polymeric sorption enhancer to modulate the elution rate of the active pharmaceutical ingredient for release of a therapeutically effective amount of the active pharmaceutical ingredient within the target range at pseudo-zero order for a period of time of at least one month. The amount of sorption enhancer may be directly proportional to the average daily elution rate.

As discussed above, the drug delivery composition may be implanted into the subject in any suitable area of the subject using any suitable means and techniques known to one of ordinary skill in the art. For example, the composition may be implanted subcutaneously, e.g., at the back of the upper arm or in the upper back (e.g., scapular region), by directly depositing in or underneath the skin, a subcutaneous fat layer, or intramuscularly.

According to another embodiment of the present invention, a kit for subcutaneously placing a drug delivery composition includes a reservoir-based drug delivery composition comprising a rate-controlling excipient defining a reservoir containing at least one discrete solid dosage form and an implanter for inserting the reservoir-based drug delivery composition beneath the skin, and optionally instructions for implantation and explanation of the drug delivery composition. The rate-controlling excipient comprises a substantially non-porous, elastomeric polymer comprising soft and hard segments and the relative content of soft and hard segments of the polymer are selected to obtain an elution rate within a target range of average daily elution rate for the active pharmaceutical ingredient. The at least one discrete solid dosage form comprises an active pharmaceutical ingredient or a pharmaceutically acceptable salt thereof and at least one non-polymeric sorption enhancer in an amount effective to modulate the elution rate of the active pharmaceutical ingredient to provide for release of the active pharmaceutical ingredient at pseudo-zero order within the target range at the therapeutically effective amount for a period of time of at least one month, and the amount of sorption enhancer may be directly proportional to the average daily elution rate.

The drug delivery composition may be delivered subcutaneously using any suitable equipment or techniques, e.g., an implanter known to one ordinary skill in the art. The kits may comprise the drug delivery composition pre-loaded into the implanter or the drug delivery composition may be loaded by the doctor or other user. The implanter may be an implantation device, such as a syringe, cannula, trocar or catheter, that may be inserted into an incision made at the delivery site of the subject. Suitable implantation devices and implantation methods include the trocar and methods disclosed in U.S. Pat. No. 7,214,206 and U.S. Pat. No. 7,510,549, the disclosures of which are herein incorporated by reference in their entirety, for all purposes. Other suitable methods for implanting or otherwise positioning the compositions into the body, e.g., by a doctor, are well known in the art. Removal and/or replacement may also be accomplished using suitable tools and methods known in the art. Kits may also comprise other equipment well known in the art, such as scalpels, clamps, suturing tools, hydration fluid, and the like.

### EXAMPLES

Embodiments of the present invention may be further understood by reference to the Examples provided below.

### Example 1

### Manufacturing of Drug Implants

Tubing was received in continuous length rolls and was cut to an appropriate starting length using a single-edged razor blade (or suitably sized scalpel). One end of each tubing section was thermally sealed imparting a semi-spherical closure on the tip of the tubing section.

The API and the salt were premixed in a Turbula blender. Stearic acid or magnesium stearate was added as a lubricant and the mixture was again mixed in a Turbula blender. The standard drug blend was 89% API, 10% salt, and 1% lubricant.

The drug blend was compacted using a single punch tablet press. Drug pellets were manually placed inside each sealed section of tubing. The open section of each pellet-containing tubing section was then sealed into a semi-spherical seal. Sterilization was accomplished by gamma irradiation of the implants.

### Example 2

### Risperidone Release from Glutamic Acid Monosodium Salt Containing Implants

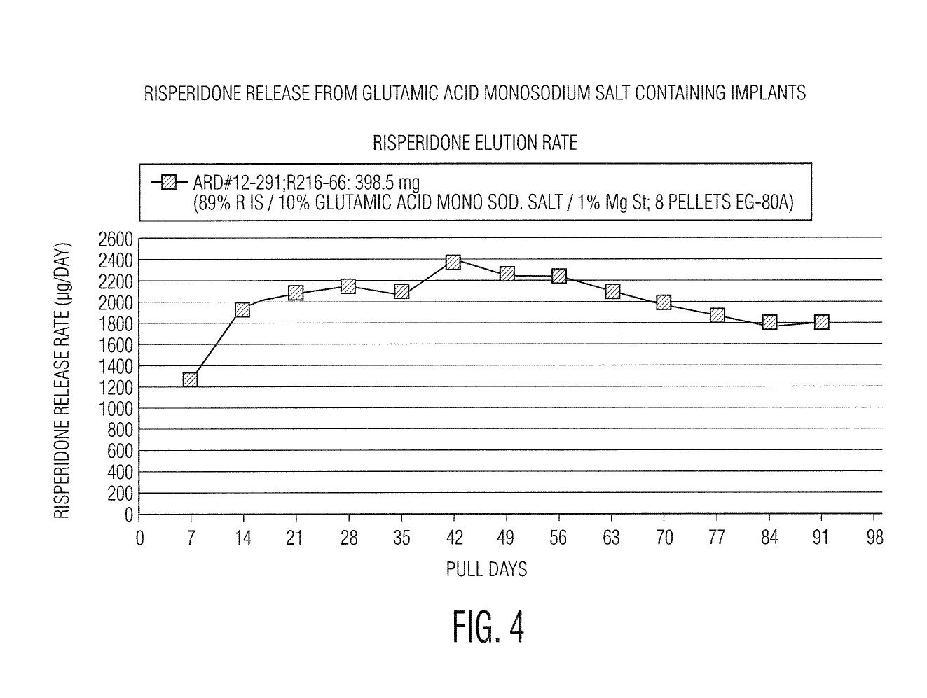

Risperidone containing pellets were manufactured as described in Example 1. Eight pellets of the drug blend were placed into Tecoflex® EG-80A polyurethane tubings of 4 mm diameter and 0.2 mm wall thickness for a total of about 400 mg risperidone per implant. The total length of each implant was about 55 mm. The glutamic acid monosodium salt (MSG) concentration was set at 10% of the final formulation. The implants were sterilized by gamma irradiation and placed in an elution bath consisting of 200 mL saline at 37° C. Weekly exchanges of the elution media were analyzed by HPLC for 12 weeks. The release graph is shown in FIG. 4. As can be seen in FIG. 4, risperidone was released from the polyurethane tubing at pseudo zero order for many weeks.

### Example 3

### Risperidone Release from Sodium Gluconate Containing Implants

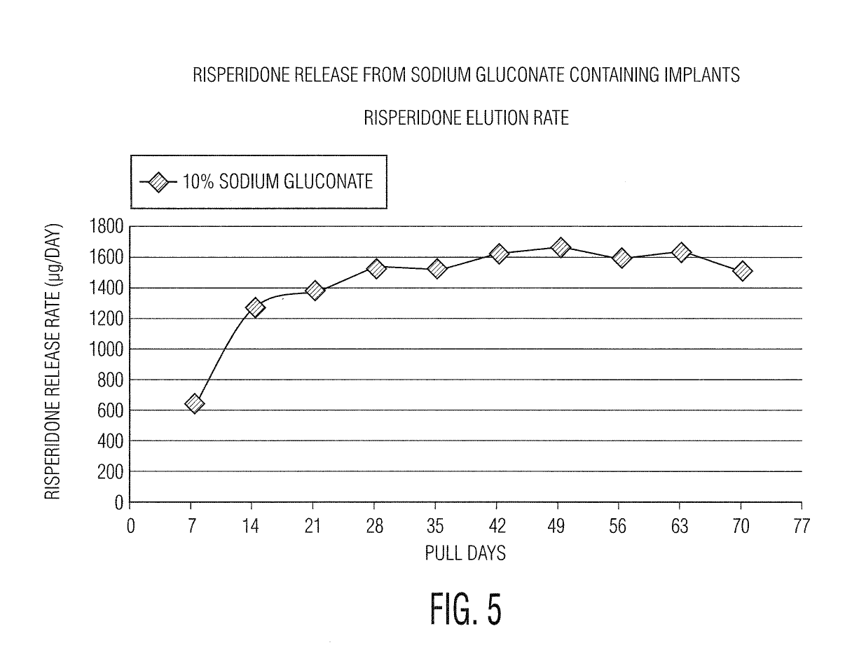

Risperidone containing pellets were manufactured as described in Example 1. Eight pellets of the drug blend were placed into Tecoflex® EG-80A polyurethane tubings of 4 mm diameter and 0.2 mm wall thickness for a total of about 400 mg risperidone per implant. The total length of each implant was about 55 mm. The sodium gluconate concentration was set at 10% of the final formulation. The implants were sterilized by gamma irradiation and placed in an elution bath consisting of 500 mL saline at 37° C. Weekly exchanges of the elution media were analyzed by HPLC for 10 weeks. The release graph is shown in FIG. 5. As can be seen in FIG. 5, risperidone was released from the polyurethane tubing at pseudo zero order for many weeks.

### Example 4

### Risperidone Release from EDTA, Potassium Sulfate and Citric Acid Containing Implants

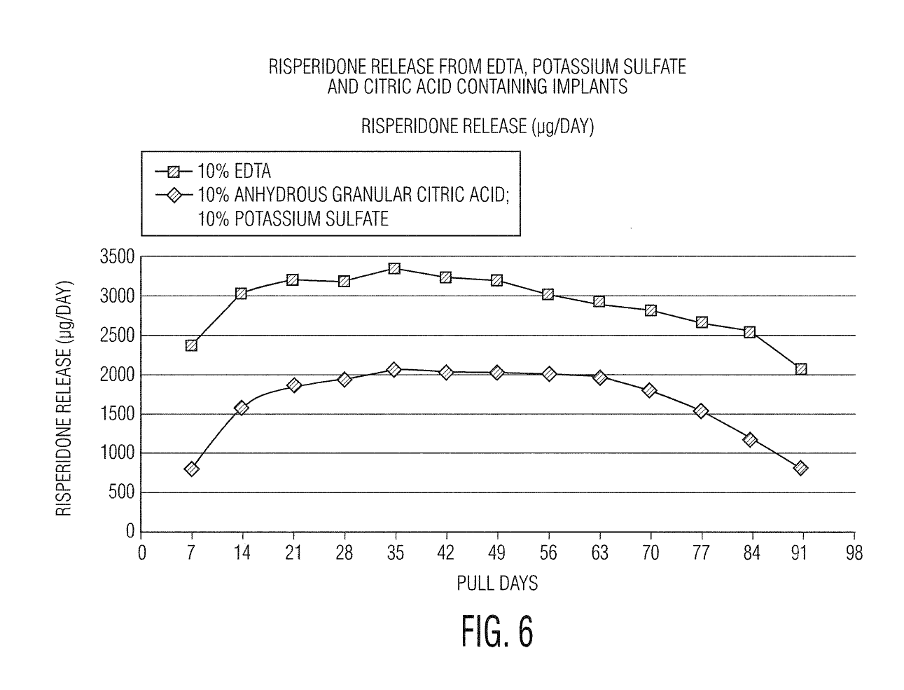

Risperidone containing pellets were manufactured as described in Example 1. Eight pellets of the drug blend were placed into Tecoflex® EG-80A polyurethane tubings of 4 mm diameter and 0.2 mm wall thickness for a total of about 475 mg risperidone per implant. The total length of each implant was about 55 mm. The concentrations of EDTA, potassium sulfate, and citric acid were each set at 10% of the final formulation. The implants were sterilized by gamma irradiation and placed in an elution bath consisting of 500 mL saline at 37° C. Weekly exchanges of the elution media were analyzed by HPLC. The release graph is shown in FIG. 6. As can be seen in FIG. 6, risperidone was released from the polyurethane tubing at pseudo zero order for many weeks.

### Example 5

### Risperidone Release from Sodium Acetate, Sodium Ascorbate, Sodium Borate, and Sodium Citrate Containing Implants

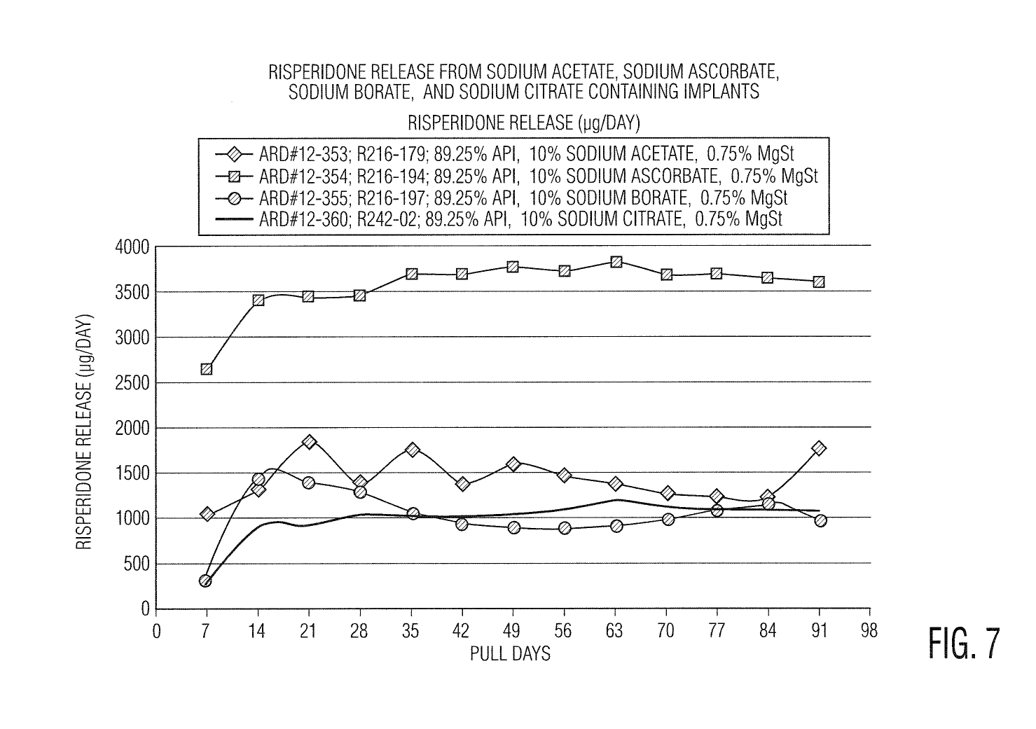

Risperidone containing pellets were manufactured as described in Example 1. Eight pellets of the drug blend were placed into Tecoflex® EG-80A polyurethane tubings of 4 mm diameter and 0.2 mm wall thickness for a total of about 475 mg risperidone per implant. The total length of each implant was about 55 mm. The concentrations of sodium acetate, sodium ascorbate, sodium borate, and sodium citrate were each set at 10% of the final formulation. The implants were sterilized by gamma irradiation and placed in an elution bath consisting of 500 mL saline at 37° C. Weekly exchanges of the elution media were analyzed by HPLC. The release graph is shown in FIG. 7. As can be seen in FIG. 7, risperidone was released from the polyurethane tubing at pseudo zero order for many weeks.

### Example 6

### Risperidone Release from Arginine and Tromethamine Containing Implants

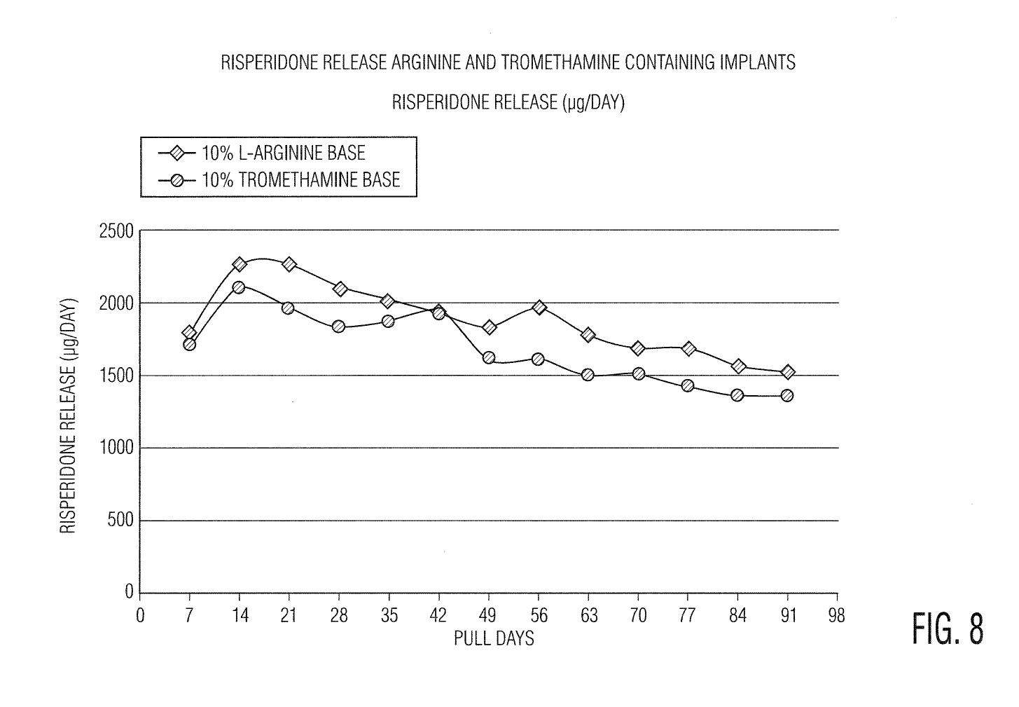

Risperidone containing pellets were manufactured as described in Example 1. Eight pellets of the drug blend were placed into Tecoflex® EG-80A polyurethane tubings of 4 mm diameter and 0.2 mm wall thickness for a total of about 480 mg risperidone per implant. The total length of each implant was about 55 mm. The concentrations of arginine and tromethamine were each set at 10% of the final formulation. The implants were sterilized by gamma irradiation and placed in an elution bath consisting of 500 mL saline at 37° C. Weekly exchanges of the elution media were analyzed by HPLC. The release graph is shown in FIG. 8. As can be seen in FIG. 8, risperidone was released from the polyurethane tubing at pseudo zero order for many weeks.

### Example 7

### Pramipexole Release from Sodium Ascorbate and EDTA Containing Implants

Pramipexole containing pellets were manufactured as described in Example 1. Eight pellets of the drug blend were placed into Tecoflex® EG-80A polyurethane tubings of 4 mm diameter and 0.2 mm wall thickness. The total length of each implant was about 50 mm. The concentrations of sodium ascorbate and EDTA were each set at 10% and 5% of the final formulation, and the pramipexole concentrations were about 400 mg and 425 mg per implant, respectively. The implants were sterilized by gamma irradiation.

### Example 8

### Vardenafil Release from Sodium Ascorbate and EDTA Containing Implants

Vardenafil containing pellets were manufactured as described in Example 1. Six pellets of the drug blend were placed into Tecoflex® EG-80A polyurethane tubings of 4 mm diameter and 0.2 mm wall thickness. The total length of each implant was about 50 mm. The concentrations of sodium ascorbate and EDTA were set at 10% of the final formulation and the vardenafil concentration was about 400 mg per implant. The implants were sterilized by gamma irradiation.

Although the invention is illustrated and described herein with reference to specific embodiments, the invention is not intended to be limited to the details shown. Rather, various modifications may be made in the details within the scope and range of equivalents of the claims and without departing from the invention.

What is claimed is:

1) A drug delivery composition comprising:
a drug elution rate-controlling excipient comprising an elastomeric polymer defining a reservoir,
wherein the reservoir contains at least one discrete solid dosage form comprising at least one active pharmaceutical ingredient (API) and one or more non-polymeric sorption enhancers,
wherein the drug delivery composition is in an implantable dosage form.

2) The drug delivery composition according to claim 1, wherein the one or more non-polymeric sorption enhancers are selected from the group consisting of acids, bases, salts, and combinations thereof.

3) The drug delivery composition according to claim 1, wherein the one or more non-polymeric sorption enhancers are selected from the group consisting of amino acids and salts thereof; citric acid and salts thereof; salts of tartaric acid, gluconic acid, acetic acid, ascorbic acid, and boric acid; polyamino carboxylic acids and salts thereof; and combinations thereof.

4) The drug delivery composition according to claim 1, wherein the one or more non-polymeric sorption enhancers are selected from the group consisting of glutamic acid monosodium salt, sodium gluconate, ethylenediaminetetraacetic acid (EDTA), potassium sulfate, citric acid, sodium acetate, sodium ascorbate, sodium borate, sodium citrate, arginine, tromethamine, sodium bitartrate, and combinations thereof.

5) The drug delivery composition according to claim 1, wherein the one or more non-polymeric sorption enhancers comprise EDTA.

6) The drug delivery composition according to claim 5, wherein the API is paliperidone.

7) The drug delivery composition according to claim 5, wherein the API is raloxifene.

8) The drug delivery composition according to claim 1, wherein the one or more non-polymeric sorption enhancers comprise sodium ascorbate.

9) The drug delivery composition according to claim 8, wherein the API is paliperidone.

10) The drug delivery composition according to claim 8, wherein the API is raloxifene.

11) The drug delivery composition according to claim 1, wherein the average molecular weight of the one or more non-polymeric sorption enhancers range from about 50 g/mol to about 400 g/mol.

12) The drug delivery composition according to claim 1, wherein the average molecular weight of the one or more non-polymeric sorption enhancers range from about 100 g/mol to about 300 g/mol.

13) The drug delivery composition according to claim 1, wherein the elastomeric polymer is substantially non-porous.

14) The drug delivery composition according to claim 1, wherein the elastomeric polymer comprises soft segments derived from polyethers, polycarbonates, or polysilicones.

15) The drug delivery composition according to claim 14, wherein the soft segments are derived from alkylene oxide polymers selected from the group consisting of poly(tetramethylene oxide) (PTMO), polyethylene glycol (PEG), poly(propylene oxide) (PPO), poly(hexamethylene oxide), and combinations thereof.

16) The drug delivery composition according to claim 1, wherein the elastomeric polymer comprises hard segments derived from polyurethanes or polyamides.

17) The drug delivery composition according to claim 1, wherein the excipient comprises an aliphatic polyether-based polyurethane comprising poly(tetramethylene oxide) and polymerized 4,4′-diisocyanato dicyclohexylmethane (H12MDI) and 1,4-butanediol.

18) The drug delivery composition according to claim 1, wherein the one or more non-polymeric sorption enhancers are present in an amount less than 30 wt % based on the total weight of the at least one discrete solid dosage form.

19) The drug delivery composition according to claim 1, wherein the one or more non-polymeric sorption enhancers are present at about 1 wt % to about 25 wt % based on the total weight of the at least one discrete solid dosage form.

20) The drug delivery composition according to claim 1, wherein the at least one API is selected from the group consisting of anastrozole, exemestane, dutasteride, oxybutynin, letrozole, selegiline, tolterodine, tizanidine, varenicline, rivastigmine, rasagiline, asenapine, paliperidone, aripiprazole, rotigotine, folic acid, vardenafil, fingolimod, laquinimod, risperidone, nicergoline, guanfacine, raloxifene, pramipexole, lidocaine, histrelin and pharmaceutically acceptable salts thereof.

21) The drug delivery composition according to claim 1, wherein the API has a molecular weight that is less than or equal to about 8,000 g/mol.

22) The drug delivery composition according to claim 1, wherein the at least one discrete solid dosage form comprises:
75-97 wt % API based on the total weight of the at least one discrete solid dosage form;
1-25 wt % of the one or more non-polymeric sorption enhancers based on the total weight of the at least one discrete solid dosage form; and
0-5 wt % lubricant based on the total weight of the at least one discrete solid dosage form.

23) A method of delivering a therapeutically effective amount of an active pharmaceutical ingredient (API) from an implantable drug delivery composition comprising:
implanting a reservoir-based drug delivery composition into a subject to systemically deliver a therapeutically effective amount of the API to the subject at a pseudo-zero order rate for a period of time of at least one month,
wherein the drug delivery composition comprises at least one discrete solid dosage form surrounded by an excipient comprising an elastomeric polymer, the at least one discrete solid dosage form comprising the API and one or more non-polymeric sorption enhancers.

24) The method according to claim 23, wherein the one or more non-polymeric sorption enhancers are selected from the group consisting of acids, bases, salts, and combinations thereof.

25) The method according to claim 23, wherein the one or more non-polymeric sorption enhancers are selected from the group consisting of amino acids and salts thereof; citric acid and salts thereof; salts of tartaric acid, gluconic acid, acetic acid, ascorbic acid, and boric acid; polyamino carboxylic acids and salts thereof; and combinations thereof.

26) The method according to claim 23, wherein the one or more non-polymeric sorption enhancers are selected from the group consisting of glutamic acid monosodium salt, sodium gluconate, ethylenediaminetetraacetic acid (EDTA), potassium sulfate, citric acid, sodium acetate, sodium ascorbate, sodium borate, sodium citrate, arginine, tromethamine, sodium bitartrate, and combinations thereof.

27) The method according to claim 23, wherein the one or more non-polymeric sorption enhancers comprise EDTA.

28) The method according to claim 23, wherein the one or more non-polymeric sorption enhancers comprise sodium ascorbate.

29) The method according to claim 23, wherein the average molecular weight of the one or more non-polymeric sorption enhancers range from about 50 g/mol to about 400 g/mol.

30) The method according to claim 23, wherein the elastomeric polymer is substantially non-porous.

31) The method according to claim 23, wherein the elastomeric polymer comprises soft segments derived from polyethers, polycarbonates, or polysilicones.

32) The method according to claim 31, wherein the soft segments are derived from alkylene oxide polymers selected from the group consisting of poly(tetramethylene oxide) (PTMO), polyethylene glycol (PEG), poly(propylene oxide) (PPO), poly(hexamethylene oxide), and combinations thereof.

33) The method according to claim 23, wherein the elastomeric polymer comprises hard segments derived from polyurethanes or polyamides.

34) The method according to claim 23, wherein the excipient comprises an aliphatic polyether-based polyurethane comprising poly(tetramethylene oxide) and polymerized 4,4′-diisocyanato dicyclohexylmethane (H12MDI) and 1,4-butanediol.

35) The method according to claim 23, wherein the one or more non-polymeric sorption enhancers are present in an amount less than 30 wt % based on the total weight of the at least one discrete solid dosage form.

36) The method according to claim 23, wherein the one or more non-polymeric sorption enhancers are present at about 1 wt % to about 25 wt % based on the total weight of the at least one discrete solid dosage form.

37) The method according to claim 23, wherein the API has a molecular weight that is less than or equal to about 8,000 g/mol.

38) The method according to claim 23, wherein the at least one discrete solid dosage form comprises:
75-97 wt % API based on the total weight of the at least one discrete solid dosage form;
1-25 wt % of the one or more non-polymeric sorption enhancers based on the total weight of the at least one discrete solid dosage form; and
0-5 wt % lubricant based on the total weight of the at least one discrete solid dosage form.

39) The method according to claim 23, wherein the API is selected from the group consisting of anastrozole, exemestane, dutasteride, oxybutynin, letrozole, selegiline, tolterodine, tizanidine, varenicline, rivastigmine, rasagiline, asenapine, paliperidone, aripiprazole, rotigotine, folic acid, vardenafil, fingolimod, laquinimod, risperidone, nicergoline, guanfacine, raloxifene, pramipexole, lidocaine, histrelin and pharmaceutically acceptable salts thereof.

40) A method of treating or preventing a disease or condition in a subject comprising:
implanting a reservoir-based drug delivery composition into a subject to systemically deliver an API to the subject for a period of time of at least one month at a pseudo-zero order elution rate,
wherein the drug delivery composition comprises at least one discrete solid dosage form surrounded by an excipient comprising an elastomeric polymer, the at least one discrete solid dosage form comprising the API and one or more non-polymeric sorption enhancers,
wherein the drug delivery composition is therapeutically effective to treat or prevent the disease or condition.

41) The method according to claim 40, wherein the disease or condition is an estrogen-related disorder and the API is an aromatase inhibitor.

42) The method according to claim 41, wherein the aromatase inhibitor is anastrozole, letrozole, exemestane, or a pharmaceutically acceptable salt thereof.

43) The method according to claim 42, wherein the estrogen-related disorder is breast cancer, endometriosis, uterine fibroids, or short stature in children.

44) The method according to claim 40, wherein the disease or condition is a psychotic disorder and the API is risperidone or a pharmaceutically acceptable salt thereof.

45) The method according to claim 44, wherein the psychotic disorder is schizophrenia, bipolar disorder, or autism.

46) The method according to claim 40, wherein the disease or condition is Parkinson's disease and the API is rasagiline or a pharmaceutically acceptable salt thereof.

47) The method according to claim 40, wherein the disease or condition is depression in a subject with Parkinson's disease and the API is rasagiline or a pharmaceutically acceptable salt thereof.

48) The method according to claim 40, wherein the disease or condition is spasticity and the API is tizanidine or a pharmaceutically acceptable salt thereof.

49) The method according to claim 40, wherein the disease or condition is osteoporosis or invasive breast cancer in a post-menopausal woman, and the API is raloxifene or a pharmaceutically acceptable salt thereof.

50) The method according to claim 40, wherein the disease or condition is Parkinson's disease and the API is pramipexole or a pharmaceutically acceptable salt thereof.

51) The method according to claim 40, wherein the disease or condition is restless legs syndrome and the API is pramipexole or a pharmaceutically acceptable salt thereof.

52) The method according to claim 40, wherein the disease or condition is pain, itch, interstitial cystitis, or overactive bladder, and the API is lidocaine or a pharmaceutically acceptable salt thereof.

53) The method according to claim 40, wherein the disease or condition is prostate cancer, uterine fibroids, or central precocious puberty, and the API is histrelin or a pharmaceutically acceptable salt thereof.

54) The method according to claim 40, wherein the disease or condition is depression, and the API is selegiline or a pharmaceutically acceptable salt thereof.

55) The method according to claim 40, wherein the one or more non-polymeric sorption enhancers are selected from the group consisting of acids, bases, salts, and combinations thereof.

56) The method according to claim 40, wherein the one or more non-polymeric sorption enhancers are selected from the group consisting of amino acids and salts thereof; citric acid and salts thereof; salts of tartaric acid, gluconic acid, acetic acid, ascorbic acid, and boric acid; polyamino carboxylic acids and salts thereof; and combinations thereof.

57) The method according to claim 40, wherein the one or more non-polymeric sorption enhancers are selected from the group consisting of glutamic acid monosodium salt, sodium gluconate, ethylenediaminetetraacetic acid (EDTA), potassium sulfate, citric acid, sodium acetate, sodium ascorbate, sodium borate, sodium citrate, arginine, tromethamine, sodium bitartrate, and combinations thereof.

58) The method according to claim 40, wherein the one or more non-polymeric sorption enhancers comprise EDTA.

59) The method according to claim 40, wherein the one or more non-polymeric sorption enhancers comprise sodium ascorbate.

60) The method according to claim 40, wherein the average molecular weight of the one or more non-polymeric sorption enhancers range from about 50 g/mol to about 400 g/mol.

61) The method according to claim 40, wherein the elastomeric polymer is substantially non-porous.

62) The method according to claim 40, wherein the elastomeric polymer comprises soft segments derived from polyethers, polycarbonates, or polysilicones.

63) The method according to claim 62, wherein the soft segments are derived from alkylene oxide polymers selected from the group consisting of poly(tetramethylene oxide) (PTMO), polyethylene glycol (PEG), poly(propylene oxide) (PPO), poly(hexamethylene oxide), and combinations thereof.

64) The method according to claim 40, wherein the elastomeric polymer comprises hard segments derived from polyurethanes or polyamides.

65) The method according to claim 40, wherein the excipient comprises an aliphatic polyether-based polyurethane comprising poly(tetramethylene oxide) and polymerized 4,4′-diisocyanato dicyclohexylmethane (H12MDI) and 1,4-butanediol.

66) The method according to claim 40, wherein the one or more non-polymeric sorption enhancers are present in an amount less than 30 wt % based on the total weight of the at least one discrete solid dosage form.

67) The method according to claim 40, wherein the one or more non-polymeric sorption enhancers are present at about 1 wt % to about 25 wt % based on the total weight of the at least one discrete solid dosage form.

68) The method according to claim 40, wherein the API has a molecular weight that is less than or equal to about 8,000 g/mol.

69) The method according to claim 40, wherein the at least one discrete solid dosage form comprises:
75-97 wt % API based on the total weight of the at least one discrete solid dosage form;
1-25 wt % of the one or more non-polymeric sorption enhancers based on the total weight of the at least one discrete solid dosage form; and
0-5 wt % lubricant based on the total weight of the at least one discrete solid dosage form.

70) The method according to claim 40, wherein the API is selected from the group consisting of anastrozole, exemestane, dutasteride, oxybutynin, letrozole, selegiline, tolterodine, tizanidine, varenicline, rivastigmine, rasagiline, asenapine, paliperidone, aripiprazole, rotigotine, folic acid, vardenafil, fingolimod, laquinimod, risperidone, nicergoline, guanfacine, raloxifene, pramipexole, lidocaine, histrelin and pharmaceutically acceptable salts thereof.

71) A subcutaneous delivery system comprising:
a thermoplastic reservoir implant comprising at least one discrete solid dosage form surrounded by a polymeric rate-controlling excipient,
the at least one discrete solid dosage form comprising at least one active pharmaceutical ingredient (API) and one or more non-polymeric sorption enhancers,
wherein the subcutaneous delivery system provides for release of the API at an elution rate suitable to provide a therapeutically effective amount of the API to a subject at a pseudo-zero order rate for a period of time of at least one month.

72) The subcutaneous delivery system according to claim 71, wherein the one or more non-polymeric sorption enhancers are selected from the group consisting of acids, bases, salts, and combinations thereof.

73) The subcutaneous delivery system according to claim 71, wherein the one or more non-polymeric sorption enhancers are selected from the group consisting of amino acids and salts thereof; citric acid and salts thereof; salts of tartaric acid, gluconic acid, acetic acid, ascorbic acid, and boric acid; polyamino carboxylic acids and salts thereof; and combinations thereof.

74) The subcutaneous delivery system according to claim 71, wherein the one or more non-polymeric sorption enhancers are selected from the group consisting of glutamic acid monosodium salt, sodium gluconate, ethylenediaminetetraacetic acid (EDTA), potassium sulfate, citric acid, sodium acetate, sodium ascorbate, sodium borate, sodium citrate, arginine, tromethamine, sodium bitartrate, and combinations thereof.

75) The subcutaneous delivery system according to claim 71, wherein the one or more non-polymeric sorption enhancers comprise EDTA.

76) The subcutaneous delivery system according to claim 71, wherein the one or more non-polymeric sorption enhancers comprise sodium ascorbate.

77) The subcutaneous delivery system according to claim 71, wherein the average molecular weight of the one or more non-polymeric sorption enhancers range from about 50 g/mol to about 400 g/mol.

78) The subcutaneous delivery system according to claim 71, wherein the elastomeric polymer is substantially non-porous.

79) The subcutaneous delivery system according to claim 71, wherein the elastomeric polymer comprises soft segments derived from polyethers, polycarbonates, or polysilicones.

80) The subcutaneous delivery system according to claim 79, wherein the soft segments are derived from alkylene oxide polymers selected from the group consisting of poly(tetramethylene oxide) (PTMO), polyethylene glycol (PEG), poly(propylene oxide) (PPO), poly(hexamethylene oxide), and combinations thereof.

81) The subcutaneous delivery system according to claim 71, wherein the elastomeric polymer comprises hard segments derived from polyurethanes or polyamides.

82) The subcutaneous delivery system according to claim 71, wherein the excipient comprises an aliphatic polyether-based polyurethane comprising poly(tetramethylene oxide) and polymerized 4,4′-diisocyanato dicyclohexylmethane (H12MDI) and 1,4-butanediol.

83) The subcutaneous delivery system according to claim 71, wherein the one or more non-polymeric sorption enhancers are present in an amount less than 30 wt % based on the total weight of the at least one discrete solid dosage form.

84) The subcutaneous delivery system according to claim 71, wherein the one or more non-polymeric sorption enhancers are present at about 1 wt % to about 25 wt % based on the total weight of the at least one discrete solid dosage form.

85) The subcutaneous delivery system according to claim 71, wherein the API has a molecular weight that is less than or equal to about 8,000 g/mol.

86) The subcutaneous delivery system according to claim 71, wherein the at least one discrete solid dosage form comprises:
75-97 wt % API based on the total weight of the at least one discrete solid dosage form;
1-25 wt % of the one or more non-polymeric sorption enhancers based on the total weight of the at least one discrete solid dosage form; and
0-5 wt % lubricant based on the total weight of the at least one discrete solid dosage form.

87) The subcutaneous delivery system according to claim 71, wherein the API is selected from the group consisting of anastrozole, exemestane, dutasteride, oxybutynin, letrozole, selegiline, tolterodine, tizanidine, varenicline, rivastigmine, rasagiline, asenapine, paliperidone, aripiprazole, rotigotine, folic acid, vardenafil, fingolimod, laquinimod, risperidone, nicergoline, guanfacine, raloxifene, pramipexole, lidocaine, histrelin and pharmaceutically acceptable salts thereof.

88) A drug delivery composition comprising:
a rate-controlling excipient defining a reservoir,
the reservoir containing at least one discrete solid dosage form comprising an active pharmaceutical ingredient,
wherein the rate-controlling excipient comprises a substantially non-porous, elastomeric polymer comprising soft and hard segments selected based on the relative content of soft and hard segments of the polymer to obtain an elution rate within a target range of average daily elution rate for the active pharmaceutical ingredient, and
the at least one discrete solid dosage form comprises one or more non-polymeric sorption enhancers in an amount effective to modulate the average daily elution rate of the active pharmaceutical ingredient to provide for release of the active pharmaceutical ingredient at pseudo-zero order within the target range at the therapeutically effective amount for a period of time of at least one month, wherein the amount of one or more non-polymeric sorption enhancers is directly proportional to the average daily elution rate.

89) The drug delivery composition according to claim 88, wherein the one or more non-polymeric sorption enhancers are selected from the group consisting of acids, bases, salts, and combinations thereof.

90) The drug delivery composition according to claim 88, wherein the one or more non-polymeric sorption enhancers are selected from the group consisting of amino acids and salts thereof; citric acid and salts thereof; salts of tartaric acid, gluconic acid, acetic acid, ascorbic acid, and boric acid; polyamino carboxylic acids and salts thereof; and combinations thereof.

91) The drug delivery composition according to claim 88, wherein the one or more non-polymeric sorption enhancers are selected from the group consisting of glutamic acid monosodium salt, sodium gluconate, ethylenediaminetetraacetic acid (EDTA), potassium sulfate, citric acid, sodium acetate, sodium ascorbate, sodium borate, sodium citrate, arginine, tromethamine, sodium bitartrate, and combinations thereof.

92) The drug delivery composition according to claim 88, wherein the one or more non-polymeric sorption enhancers comprise EDTA.

93) The drug delivery composition according to claim 88, wherein the one or more non-polymeric sorption enhancers comprise sodium ascorbate.

94) The drug delivery composition according to claim 88, wherein the average molecular weight of the one or more non-polymeric sorption enhancers range from about 50 g/mol to about 400 g/mol.

95) The drug delivery composition according to claim 88, wherein the one or more non-polymeric sorption enhancers are present in an amount less than 30 wt % based on the total weight of the at least one discrete solid dosage form.

96) The drug delivery composition according to claim 88, wherein the one or more non-polymeric sorption enhancers are present at about 1 wt % to about 25 wt % based on the total weight of the at least one discrete solid dosage form.

97) The drug delivery composition according to claim 88, wherein the API has a molecular weight that is less than or equal to about 8,000 g/mol.

98) The drug delivery composition according to claim 88, wherein the at least one discrete solid dosage form comprises:
75-97 wt % API based on the total weight of the at least one discrete solid dosage form;
1-25 wt % of the one or more non-polymeric sorption enhancers based on the total weight of the at least one discrete solid dosage form; and
0-5 wt % lubricant based on the total weight of the at least one discrete solid dosage form.

99) The drug delivery composition according to claim 88, wherein the API is selected from the group consisting of anastrozole, exemestane, dutasteride, oxybutynin, letrozole, selegiline, tolterodine, tizanidine, varenicline, rivastigmine, rasagiline, asenapine, paliperidone, aripiprazole, rotigotine, folic acid, vardenafil, fingolimod, laquinimod, risperidone, nicergoline, guanfacine, raloxifene, pramipexole, lidocaine, histrelin and pharmaceutically acceptable salts thereof.

100) A kit for subcutaneously placing a drug delivery composition comprising:
a reservoir-based drug delivery composition comprising a rate-controlling excipient defining a reservoir containing at least one discrete solid dosage form, the at least one discrete solid dosage form comprising an API and one or more non-polymeric sorption enhancers; and
an implanter for inserting the reservoir-based drug delivery composition beneath the skin.

101) The kit according to claim 100, wherein the one or more non-polymeric sorption enhancers are selected from the group consisting of acids, bases, salts, and combinations thereof.

102) The kit according to claim 100, wherein the one or more non-polymeric sorption enhancers are selected from the group consisting of amino acids and salts thereof; citric acid and salts thereof; salts of tartaric acid, gluconic acid, acetic acid, ascorbic acid, and boric acid; polyamino carboxylic acids and salts thereof; and combinations thereof.

103) The kit according to claim 100, wherein the one or more non-polymeric sorption enhancers are selected from the group consisting of glutamic acid monosodium salt, sodium gluconate, ethylenediaminetetraacetic acid (EDTA), potassium sulfate, citric acid, sodium acetate, sodium ascorbate, sodium borate, sodium citrate, arginine, tromethamine, sodium bitartrate, and combinations thereof.

104) The kit according to claim 100, wherein the one or more non-polymeric sorption enhancers comprise EDTA.

105) The kit according to claim 100, wherein the one or more non-polymeric sorption enhancers comprise sodium ascorbate.

106) The kit according to claim 100, wherein the average molecular weight of the one or more non-polymeric sorption enhancers range from about 50 g/mol to about 400 g/mol.

107) The kit according to claim 100, wherein the one or more non-polymeric sorption enhancers are present in an amount less than 30 wt % based on the total weight of the at least one discrete solid dosage form.

108) The kit according to claim 100, wherein the one or more non-polymeric sorption enhancers are present at about 1 wt % to about 25 wt % based on the total weight of the at least one discrete solid dosage form.

109) The kit according to claim 100, wherein the API has a molecular weight that is less than or equal to about 8,000 g/mol.

110) The kit according to claim 100, wherein the at least one discrete solid dosage form comprises:
75-97 wt % API based on the total weight of the at least one discrete solid dosage form;
1-25 wt % of the one or more non-polymeric sorption enhancers based on the total weight of the at least one discrete solid dosage form; and
0-5 wt % lubricant based on the total weight of the at least one discrete solid dosage form.

111) The kit according to claim 100, wherein the API is selected from the group consisting of anastrozole, exemestane, dutasteride, oxybutynin, letrozole, selegiline, tolterodine, tizanidine, varenicline, rivastigmine, rasagiline, asenapine, paliperidone, aripiprazole, rotigotine, folic acid, vardenafil, fingolimod, laquinimod, risperidone, nicergoline, guanfacine, raloxifene, pramipexole, lidocaine, histrelin and pharmaceutically acceptable salts thereof.

112) The drug delivery composition according to claim 1, wherein the one or more non-polymeric sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

113) The method according to claim 23, wherein the one or more non-polymeric sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

114) The method according to claim 40, wherein the one or more non-polymeric sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

115) The subcutaneous delivery system according to claim 71, wherein the one or more non-polymeric sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

116) The drug delivery composition according to claim 88, wherein the one or more non-polymeric sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

117) The kit according to claim 100, wherein the one or more non-polymeric sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

118) The drug delivery composition according to claim 1, wherein the at least one discrete solid dosage form further comprises one or more additional sorption enhancers selected from the group consisting of one or more sugar-based sorption enhancers, one or more polymeric sorption enhancers, and a combination thereof.

119) The drug delivery composition according to claim 118, wherein the sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

120) The method according to claim 23, wherein the at least one discrete solid dosage form further comprises one or more additional sorption enhancers selected from the group consisting of one or more sugar-based sorption enhancers, one or more polymeric sorption enhancers, and a combination thereof.

121) The method according to claim 120, wherein the sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

122) The method according to claim 40, wherein the at least one discrete solid dosage form further comprises one or more additional sorption enhancers selected from the group consisting of one or more sugar-based sorption enhancers, one or more polymeric sorption enhancers, and a combination thereof.

123) The method according to claim 122, wherein the sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

124) The subcutaneous delivery system according to claim 71, wherein the at least one discrete solid dosage form further comprises one or more additional sorption enhancers selected from the group consisting of one or more sugar-based sorption enhancers, one or more polymeric sorption enhancers, and a combination thereof.

125) The subcutaneous delivery system according to claim 124, wherein the sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

126) The drug delivery composition according to claim 88, wherein the at least one discrete solid dosage form further comprises one or more additional sorption enhancers selected from the group consisting of one or more sugar-based sorption enhancers, one or more polymeric sorption enhancers, and a combination thereof.

127) The drug delivery composition according to claim 126, wherein the sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

128) The kit according to claim 100, wherein the at least one discrete solid dosage form further comprises one or more additional sorption enhancers selected from the group consisting of one or more sugar-based sorption enhancers, one or more polymeric sorption enhancers, and a combination thereof.

129) The kit according to claim 128, wherein the sorption enhancers comprise a combination of a first sorption enhancer which produces a first average daily elution rate in the drug delivery composition and a second sorption enhancer which produces a second average daily elution rate in the drug delivery composition, wherein the second average daily elution rate is less than the first average daily elution rate, and wherein the combination of the first and second sorption enhancers produces an average daily elution rate that is intermediate between the first and second average daily elution rates.

130) The drug delivery composition according to claim 1, wherein the one or more non-polymeric sorption enhancers are selected from the group consisting of ascorbic acid and analogs or derivatives thereof; gallic acid and analogs or derivatives thereof; and combinations thereof.

131) The drug delivery composition according to claim 1, wherein the one or more non-polymeric sorption enhancers are selected from the group consisting of ascorbic acid, erythorbic acid, dehydroascorbic acid, calcium ascorbate dihydrate, ascorbyl palmitate, sodium ascorbyl phosphate, gallic acid, n-octyl gallate, propyl gallate, and combinations thereof.

132) The method of claim 23, wherein the one or more non-polymeric sorption enhancers are selected from the group consisting of ascorbic acid and analogs or derivatives thereof; gallic acid and analogs or derivatives thereof; and combinations thereof.

133) The method of claim 23, wherein the one or more non-polymeric sorption enhancers are selected from the group consisting of ascorbic acid, erythorbic acid, dehydroascorbic acid, calcium ascorbate dihydrate, ascorbyl palmitate, sodium ascorbyl phosphate, gallic acid, n-octyl gallate, propyl gallate, and combinations thereof.

134) The method of claim 40, wherein the one or more non-polymeric sorption enhancers are selected from the group consisting of ascorbic acid and analogs or derivatives thereof; gallic acid and analogs or derivatives thereof; and combinations thereof.

135) The method of claim 40, wherein the one or more non-polymeric sorption enhancers are selected from the group consisting of ascorbic acid, erythorbic acid, dehydroascorbic acid, calcium ascorbate dihydrate, ascorbyl palmitate, sodium ascorbyl phosphate, gallic acid, n-octyl gallate, propyl gallate, and combinations thereof.

136) The subcutaneous delivery system according to claim 71, wherein the one or more non-polymeric sorption enhancers are selected from the group consisting of ascorbic acid and analogs or derivatives thereof; gallic acid and analogs or derivatives thereof; and combinations thereof.

137) The subcutaneous delivery system according to claim 71, wherein the one or more non-polymeric sorption enhancers are selected from the group consisting of ascorbic acid, erythorbic acid, dehydroascorbic acid, calcium ascorbate dihydrate, ascorbyl palmitate, sodium ascorbyl phosphate, gallic acid, n-octyl gallate, propyl gallate, and combinations thereof.

138) The drug delivery composition according to claim 88, wherein the one or more non-polymeric sorption enhancers are selected from the group consisting of ascorbic acid and analogs or derivatives thereof; gallic acid and analogs or derivatives thereof; and combinations thereof.

139) The drug delivery composition according to claim 88, wherein the one or more non-polymeric sorption enhancers are selected from the group consisting of ascorbic acid, erythorbic acid, dehydroascorbic acid, calcium ascorbate dihydrate, ascorbyl palmitate, sodium ascorbyl phosphate, gallic acid, n-octyl gallate, propyl gallate, and combinations thereof.

140) The kit according to claim 100, wherein the one or more non-polymeric sorption enhancers are selected from the group consisting of ascorbic acid and analogs or derivatives thereof; gallic acid and analogs or derivatives thereof; and combinations thereof.

141) The kit according to claim 100, wherein the one or more non-polymeric sorption enhancers are selected from the group consisting of ascorbic acid, erythorbic acid, dehydroascorbic acid, calcium ascorbate dihydrate, ascorbyl palmitate, sodium ascorbyl phosphate, gallic acid, n-octyl gallate, propyl gallate, and combinations thereof.
